(19) United States
(12) Patent Application Publication
(10) Pub. No.: US 2026/0028610 A1
(43) Pub. Date: Jan. 29, 2026

(54) IMPROVED IGG-DEGRADING ENZYMES AND METHODS OF USE THEREOF

(71) Applicant: AstraZeneca Ireland Limited, Dublin (IE)

(72) Inventors: Joanne Irene BRODFUEHRER, Cambridge, MA (US); Amendra Fernando Hewa DEWAGE, Cambridge, MA (US); Hannah Szydlo GARDNER, Arlington, MA (US); Alison Patricia JOYCE, Cambridge, MA (US); Amy Cotsonas KING, Cambridge, MA (US); Maria Paquerette Galou LAMEYER, Cambridge, MA (US); Kok Hong LIM, Malden, MA (US); Laura Lin LOHSE, Cambridge, MA (US); Nicholas Andrew MARZE, Cambridge, MA (US); Denise Mary MURPHY, Swampscott, MA (US); Alyssa Ann MYERS, Cambridge, MA (US); Sze Pui TAM, Cambridge, MA (US)

(73) Assignee: AstraZeneca Ireland Limited, Dublin (IE)

(21) Appl. No.: 18/846,588

(22) PCT Filed: Mar. 14, 2023
(86) PCT No.: PCT/IB2023/052463

§ 371 Date: Sep. 12, 2024

Related U.S. Application Data
(60) Provisional application No. 63/269,502, filed on Mar. 17, 2022.
(60) Provisional application No. 63/483,984, filed on Feb. 9, 2023.
(60) Provisional application No. 63/485,312, filed on Feb. 16, 2023.

(51) Int. Cl. C12N 9/64; A61K 9/00; A61K 38/00; A61K 48/00; A61P 37/06; C12N 15/52; C12N 15/86

(52) U.S. Cl. CPC C12N 9/641; A61K 9/0019; A61K 48/0008; A61K 48/0083; A61P 37/06; C12N 15/52; C12N 15/86; A61K 38/00; C12N 2710/10043; C12N 2740/15043; C12N 2750/14143; C12Y 304/2201

(57) ABSTRACT

Provided are improved cysteine proteases for specifically cleaving and inactivating immunoglobulin G. The improved cysteine pro-teases are useful in methods of treating diseases, disorders or conditions characterized by excessive levels of IgG antibodies, including autoimmune disorders and candidates for organ transplantation sensitized with anti-HLA antibodies, as well as for treating candidates for gene therapy with pre-existing neutralizing antibodies against recombinant vectors and re-dosing of subjects previously treated with a gene therapy vector.

The instant application contains a Sequence Listing which has been submitted electronically in XML format and is hereby incorporated by reference in its entirety. Said XML copy, created on Oct. 15, 2025 is named 51744-040001_Sequence_Listing_10_15_25_ST26.xml and is 100,646 bytes in size.

### BACKGROUND

Immunoglobulin G-degrading enzyme of Streptococcus pyogenes (IdeS) is a naturally occurring cysteine protease expressed by the pathogenic bacteria Streptococcus pyogenes which exhibits specificity for its target sequence found in human IgG, in addition to several other species. IdeS is capable of cleaving IgG below the hinge region, leading to the generation of F(ab′)2 and Fc/2 fragments. IdeS is capable of cleaving IgG in human plasma and can reduce total IgG levels in humans shortly after its administration.

Certain disorders and diseases of humans are mediated by IgG antibodies, notably autoimmune disorders such as diabetes type 1 and multiple sclerosis, causing untold suffering. Further, presence of IgG antibodies can thwart successful administration of established life saving therapies, such as organ transplantation, as well as more recently developed treatments, such as gene therapy using recombinant viral vectors. A variety of methods have been attempted to mitigate the deleterious effect of such pathogenic IgG antibodies in patients with autoimmune disorders, and candidates for transplantation and gene therapy, but with incomplete efficacy.

Thus, there exists a need in the art for compositions and methods capable of degrading, digesting, and inactivating pathogenic IgG antibodies in subjects, particularly human subjects with autoimmune disorders, or who are candidates for transplantation or gene therapy. And, related to that need, there also exists a need for IdeS protein variants having improved potency and/or stability.

### SUMMARY

Those skilled in the art will recognize or will be able to ascertain using no more than routine experimentation, many equivalents to the specific embodiments described herein. Such equivalents are intended to be encompassed by the following embodiments (E).
E1. An isolated cysteine protease that specifically cleaves immunoglobulin G (IgG) antibody molecules.
E2. The cysteine protease of E1, wherein said protease has greater potency or thermal stability relative to wildtype IdeS.
E3. The cysteine protease of E2, wherein said cysteine protease has a Tₒₙₛₑₜ value, as determined using differential scanning calorimetry, that is at least or about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4.0° C. greater compared to wildtype IdeS protein.
E4. The cysteine protease of E2, wherein said cysteine protease has a Tₒₙₛₑₜ value, as determined using differential scanning calorimetry, that is at least or about 44.0, 44.1, 44.2, 44.3, 44.4, 44.5, 44.6, 44.7, 44.8, 44.9, 45.0, 45.1, 45.2, 45.3, 45.4, 45.5, 45.6, 45.7, 45.8, 45.9, 46.0, 46.1, 46.2, 46.3, 46.4, 46.5, 46.6, 46.7, 46.8, 46.9, 47.0, 47.1, 47.2, 47.3, 47.4, 47.5, 47.6, 47.7, 47.8, 47.9, 48.0, 48.1, 48.2, 48.3, 48.4, 48.5, 48.6, 48.7, 48.8, 48.9, 49.0, 49.1, 49.2, 49.3, 49.4, 49.5, 49.6, 49.7, 49.8, 49.9, or 50.0° C.
E5. The cysteine protease of E2, wherein said cysteine protease has a TM value, as determined using differential scanning calorimetry, that is at least or about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4.0° C. greater compared to wildtype IdeS protein.
E6. The cysteine protease of E2, wherein said cysteine protease has a TM value, as determined using differential scanning calorimetry, that is at least or about 51.0, 51.1, 51.2, 51.3, 51.4, 51.5, 51.6, 51.7, 51.8, 51.9, 52.0, 52.1, 52.2, 52.3, 52.4, 52.5, 52.6, 52.7, 52.8, 52.9, 53.0, 53.1, 53.2, 53.3, 53.4, 53.5, 53.6, 53.7, 53.8, 53.9, 54.0, 54.1, 54.2, 54.3, 54.4, 54.5, 54.6, 54.7, 54.8, 54.9, 55.0, 55.1, 55.2, 55.3, 55.4, 55.5, 55.6, 55.7, 55.8, 55.9, 56.0, 56.1, 56.2, 56.3, 56.4, 56.5, 56.6, 56.7, 56.8, 56.9, or 57.0° C.
E7. The cysteine protease of E2, wherein said cysteine protease has IgG cleaving potency, as determined using ELISA and expressed as an IC50 value, that is at least or about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, or 2.5 nM less compared to wildtype IdeS.

The cysteine protease of E2, wherein said cysteine protease has IgG cleaving potency, as determined using ELISA and expressed as an IC50 value, that is at most 5.5, 5.4, 5.3, 5.2, 5.1, 5.0, 4.9, 4.8, 4.7, 4.6, 4.5, 4.4, 4.3, 4.2, 4.1, 4.0, 3.9, 3.8, or 3.7 nM.
E8. The cysteine protease of E2, wherein the amino acid sequence of said cysteine protease comprises, consists essentially of, or consists of amino acid numbers 1-312 of any one of SEQ ID NOs: 2-71, or amino acid numbers 2-312 of any one of SEQ ID NOs: 2-71, or amino acid numbers 3-312 of any one of SEQ ID NOs: 3-71.
E9. The cysteine protease of E9, wherein the amino acid sequence of said cysteine protease comprises, consists essentially of, or consists of the amino acid sequence of any one of SEQ ID NOs: 72-82.
E10. A pharmaceutical composition comprising the cysteine protease of any one of embodiments E1 to E10 and a pharmaceutically acceptable carrier.
E11. A method of treating a subject in need of treatment or prevention of a disease or disorder characterized by an excess of IgG antibodies, comprising administering to said subject an amount of the cysteine protease of E10 that is effective to reduce the concentration of IgG antibodies in a bodily fluid of said subject.
E12. The method of E12, wherein the bodily fluid is blood, plasma, or serum.
E13. The method of E12, wherein said cysteine protease acts by degrading, digesting, or inactivating said IgG antibodies.
E14. The method of E12, wherein the disease or disorder is an autoimmune disease or disorder, and wherein administering said cysteine protease is effective to treat or prevent said autoimune disease or disorder.
E15. The method of E15, wherein an effective amount of said cysteine protease is a dose of at least or about 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.35, 0.40, 0.45, or 0.50 mg/kg subject body weight.
E16. The method of E15, wherein said treatment is effective to reduce the concentration of total IgG in the subject's bodily fluid by at least or about 60%, 65%, 70%, 75%, 80%, 85%, 90%, 91%, 92%, 93%, 94%, 95%, 96%, 97%, 98%, 99%, or 100%.
E17. The method of E15, wherein said treatment is effective to reduce the concentration of total IgG in the subject's bodily fluid to not more than about 7, 6.5, 6, 5.5, 5, 4.5, 4, 3.5, 3, 2.5, 2.4, 2.3, 2.2, 2.1, 2.0, 1.9, 1.8, 1.7, 1.6, 1.5, 1.4, 1.3, 1.2, 1.1, 1.0, 0.9, 0.8, 0.7, 0.6, 0.5, 0.4, 0.3, 0.2, 0.1, 0.05 grams per liter.
E18. A method of treating a sensitized subject in need of transplantation of a tissue or organ, comprising administering to said subject an amount of the cysteine protease of E10 that is effective to reduce the concentration of anti-HLA antibodies in a bodily fluid of said subject sufficiently to prevent antibody-mediated rejection of said tissue or organ after transplantation.
E19. The method of E19, wherein the bodily fluid is blood, plasma, or serum.
E20. The method of E19, wherein said cysteine protease acts by degrading, digesting, or inactivating said anti-HLA antibodies.
E21. The method of E19, wherein the organ is kidney, liver, heart, pancreas, lung, or intestine.
E22. The method of E19, wherein said subject exhibits a calculated panel reactive antibody assay score of at least or about 10%, 15%, 20%, 25%, 30%, 35%, 40%, 45%, 50%, 55%, or 60%.
E23. The method of E19, wherein an effective amount of said cysteine protease is a dose of at least or about 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.35, 0.40, 0.45, or 0.50 mg/kg subject body weight.
E24. The method of E19, wherein said treatment is effective to reduce the concentration of anti-HLA antibodies in the subject's bodily fluid by at least or about 60%, 65%, 70%, 75%, 80%, 85%, 90%, 91%, 92%, 93%, 94%, 95%, 96%, 97%, 98%, 99%, or 100%.
E25. The method of E19, wherein said treatment is effective to reduce the concentration of total IgG in the subject's bodily fluid to not more than about 7, 6.5, 6, 5.5, 5, 4.5, 4, 3.5, 3, 2.5, 2.4, 2.3, 2.2, 2.1, 2.0, 1.9, 1.8, 1.7, 1.6, 1.5, 1.4, 1.3, 1.2, 1.1, 1.0, 0.9, 0.8, 0.7, 0.6, 0.5, 0.4, 0.3, 0.2, 0.1, 0.05 grams per liter.
E26. The method of E19, wherein said treatment is effective to reduce the calculated panel reactive antibody assay score of said subject's serum by at least or about 50%, 55%, 60%, 65%, 70%, 75%, 80%, 85%, 90%, or 95%.
E27. The method of E19, wherein said treatment is effective for the calculated panel reactive antibody assay score of said subject's serum to be reduced to not more than about 50%, 45%, 40%, 35%, 30%, 25%, 20%, 19%, 18%, 17%, 16%, 15%, 14%, 13%, 12%, 11%, 10%, 9%, 8%, 7%, 6%, 5%, 4%, 3%, 2%, or 1%.
E28. The method of E19, wherein the subject subsequently undergoes tissue or organ transplantation, and the period between administering the cysteine protease and subsequent transplantation is at least or about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 hours.
E29. A method of treating a subject in need of therapy with a gene therapy vector, comprising administering to said subject an amount of the cysteine protease of E10 that is effective to reduce the concentration of IgG antibodies in a bodily fluid of said subject that are specific for a component of said gene therapy vector.
E30. The method of E30, wherein the bodily fluid is blood, plasma, or serum.
E31. The method of E30, wherein said cysteine protease acts by degrading, digesting, or inactivating said IgG antibodies.
E32. The method of E30, wherein said IgG antibodies are neutralizing antibodies.
E33. The method of E30, wherein said gene therapy vector is a recombinant viral vector.
E34. The method of E32, wherein said recombinant viral vector is a recombinant adenoviral vector, recombinant adeno-associated viral vector, or recombinant lentiviral vector.
E35. The method of E33, wherein the titer of neutralizing antibodies is at least or about 1:2, 1:3, 1:4, 1:5, 1:6, 1:7, 1:8, 1:9, 1:10, 1:15, 1:20, 1:25, 1:30, 1:40, 1:50, 1:60, 1:70, 1:80, 1:90, 1:100, 1:120, 1:125, 1:150, 1:175, 1:200, 1:250, 1:300, 1:350, 1:400, 1:450, 1:500, 1:550, 1:600, 1:650, 1:700, 1:750, 1:800, 1:850, 1:900, 1:950, 1:1000, 1:1100, 1:1200, 1:1300, 1:1400, 1:1500, 1:1600, 1:1700, 1:1800, 1:1900, 1:2000, 1:2100, 1:2200, 1:2300, 1:2400, 1:2500, 1:2600, 1:2700, 1:2800, 1:2900, or 1:3000.
E36. The method of E33, wherein an effective amount of said cysteine protease is a dose of at least or about 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.35, 0.40, 0.45, or 0.50 mg/kg subject body weight.
E37. The method of E33, wherein said treatment is effective to reduce the titer of neutralizing antibodies in the subject's bodily fluid by at least or about 60%, 65%, 70%, 75%, 80%, 85%, 90%, 91%, 92%, 93%, 94%, 95%, 96%, 97%, 98%, 99%, or 100%.
E38. The method of E33, wherein said treatment is effective to reduce the reduce the titer neutralizing antibodies in the subject's bodily fluid to a value of not more than 1:10, 1:9, 1:8, 1:7, 1:6, 1:5, 1:4, 1:3, 1:2, 1:1, 1:0.9, 1:0.8, 1:0.7, 1:0.6, 1:0.5, 1:0.4, 1:0.3, 1:0.2, or 1:0.1.
E39. The method of E33, wherein said subject is gene therapy treatment naïve.
E40. The method of E40, wherein the subject subsequently undergoes treatment with said gene therapy vector.
E41. The method of E41, wherein said gene therapy vector is a recombinant viral vector.
E42. The method of E42, wherein said recombinant viral vector is a recombinant adenoviral vector, recombinant adeno-associated viral vector, or recombinant lentiviral vector.
E43. The method of E41, wherein the period between administering said cysteine protease and subsequent gene therapy is at least or about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 50, 52, 54, 55, 56, 58, 60, 62, 64, 65, 66, 68, 70, 72, 74, 75, 76, 78, 80, 84, 85, 90, 95, 96, 100, 108, 110, 120, 125, 130, 132, 135, 140, 144, 145, 150, 155, 156, 160, 165, or 168 hours, or 8, 9, 10, 11, 12, 13, or 14 days.
E44. The method of E41, wherein the titer of neutralizing antibodies is at least or about 1:2, 1:3, 1:4, 1:5, 1:6, 1:7, 1:8, 1:9, 1:10, 1:15, 1:20, 1:25, 1:30, 1:40, 1:50, 1:60, 1:70, 1:80, 1:90, 1:100, 1:120, 1:125, 1:150, 1:175, 1:200, 1:250, 1:300, 1:350, 1:400, 1:450, 1:500, 1:550, 1:600, 1:650, 1:700, 1:750, 1:800, 1:850, 1:900, 1:950, 1:1000, 1:1100, 1:1200, 1:1300, 1:1400, 1:1500, 1:1600, 1:1700, 1:1800, 1:1900, 1:2000, 1:2100, 1:2200, 1:2300, 1:2400, 1:2500, 1:2600, 1:2700, 1:2800, 1:2900, or 1:3000.
E45. The method of E41, wherein an effective amount of said cysteine protease is a dose of at least or about 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.35, 0.40, 0.45, or 0.50 mg/kg subject body weight.
E46. The method of E41, wherein said treatment is effective to reduce the titer of neutralizing antibodies in the subject's bodily fluid by at least or about 60%, 65%, 70%, 75%, 80%, 85%, 90%, 91%, 92%, 93%, 94%, 95%, 96%, 97%, 98%, 99%, or 100%.
E47. The method of E41, wherein said treatment is effective to reduce the reduce the titer neutralizing antibodies in the subject's bodily fluid to a value of not more than 1:10, 1:9, 1:8, 1:7, 1:6, 1:5, 1:4, 1:3, 1:2, 1:1, 1:0.9, 1:0.8, 1:0.7, 1:0.6, 1:0.5, 1:0.4, 1:0.3, 1:0.2, or 1:0.1.
E48. The method of E33, wherein said subject was treated at least once previously with the same type of gene therapy vector with which said subject is in need of therapy.
E49. The method of E49, wherein the subject subsequently undergoes treatment with said gene therapy vector.
E50. The method of E50, wherein said gene therapy vector is a recombinant viral vector.
E51. The method of E51, wherein said recombinant viral vector is a recombinant adenoviral vector, recombinant adeno-associated viral vector, or recombinant lentiviral vector.
E52. The method of E50, wherein the period between administering said cysteine protease and subsequent gene therapy is at least or about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 50, 52, 54, 55, 56, 58, 60, 62, 64, 65, 66, 68, 70, 72, 74, 75, 76, 78, 80, 84, 85, 90, 95, 96, 100, 108, 110, 120, 125, 130, 132, 135, 140, 144, 145, 150, 155, 156, 160, 165, or 168 hours, or 8, 9, 10, 11, 12, 13, or 14 days.
E53. The method of E50, wherein the titer of neutralizing antibodies is at least or about 1:2, 1:3, 1:4, 1:5, 1:6, 1:7, 1:8, 1:9, 1:10, 1:15, 1:20, 1:25, 1:30, 1:40, 1:50, 1:60, 1:70, 1:80, 1:90, 1:100, 1:120, 1:125, 1:150, 1:175, 1:200, 1:250, 1:300, 1:350, 1:400, 1:450, 1:500, 1:550, 1:600, 1:650, 1:700, 1:750, 1:800, 1:850, 1:900, 1:950, 1:1000, 1:1100, 1:1200, 1:1300, 1:1400, 1:1500, 1:1600, 1:1700, 1:1800, 1:1900, 1:2000, 1:2100, 1:2200, 1:2300, 1:2400, 1:2500, 1:2600, 1:2700, 1:2800, 1:2900, or 1:3000.
E54. The method of E50, wherein an effective amount of said cysteine protease is a dose of at least or about 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.35, 0.40, 0.45, or 0.50 mg/kg subject body weight.
E55. The method of E50, wherein said treatment is effective to reduce the titer of neutralizing antibodies in the subject's bodily fluid by at least or about 60%, 65%, 70%, 75%, 80%, 85%, 90%, 91%, 92%, 93%, 94%, 95%, 96%, 97%, 98%, 99%, or 100%.
E56. The method of E50, wherein said treatment is effective to reduce the reduce the titer neutralizing antibodies in the subject's bodily fluid to a value of not more than 1:10, 1:9, 1:8, 1:7, 1:6, 1:5, 1:4, 1:3, 1:2, 1:1, 1:0.9, 1:0.8, 1:0.7, 1:0.6, 1:0.5, 1:0.4, 1:0.3, 1:0.2, or 1:0.1.
E57. The method of any one of embodiments E12 to E57, wherein said subject is a human subject.
E58. The method of any one of embodiments E12 to E58, wherein said cysteine protease is administered parenterally.
E59. The method of E59, wherein said cysteine protease is administered intravenously or intra-arterially.
E60. The method of any one of embodiments E12, E19, or E30, wherein said step of administering the cysteine protease to said subject is repeated at least one time.
E61. A kit comprising a container having disposed therein a pharmaceutical composition comprising a cysteine protease, and a label with instructions for performing a method according to any one of embodiments E12 to E61.
E62. A polynucleotide encoding the cysteine protease of any one of embodiments E1 to E10.
E63. An expression vector comprising the polynucleotide of E63.
E64. A host cell comprising the expression vector of E64.
E65. The host cell of E65, wherein said host cell is a bacterial host cell.
E66. A method for making a cysteine protease comprising incubating the host cell of E66 under conditions sufficient to express said cysteine protease and purifying the cysteine protease produced thereby.

### BRIEF DESCRIPTION OF THE DRAWINGS

FIGS. 1A-1J depict alignment of the amino acid sequences of wildtype IdeS and IdeS variant proteins created by protein engineering based on the published IdeS crystal structure. Among the variant sequences, highlighted residues differ from those in the wildtype sequence.

FIG. 2 depicts a bar graph summarizing stability data for certain IdeS variants compared to wildtype IdeS (GBT-NCC-0005).

FIG. 3A depicts a Coomassie stained polyacrylamide gel illustrating protein fragments resulting from digesting IgG protein with wildtype IdeS and certain IdeS variants.

FIG. 3B depicts a Coomassie stained polyacrylamide gel illustrating protein fragments resulting from digesting IgM protein with wildtype IdeS and certain IdeS variants.

FIG. 4 depicts a schematic of the MSD assay format.

FIG. 5 depicts a graph summarizing amounts of intact IgG (% of baseline) in rabbits treated with wildtype IdeS (WT) versus an IdeS variant (Var).

FIG. 6 depicts a schematic of the MSD LBA assay format.

FIG. 7 depicts a graph summarizing mean PK of an IdeS variant (triangle) and wildtype Ides (square).

### DETAILED DESCRIPTION

### General Techniques

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-1998) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C.A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995).

### Definitions

Unless otherwise defined, all technical and scientific terms used herein have the same meaning as commonly understood by one of ordinary skill in the art to which this invention belongs. In case of conflict, the present specification, including definitions, will control. Unless otherwise required by context, singular terms shall include pluralities and plural terms shall include the singular. Any example(s) following the term “e.g.” or “for example” is not meant to be exhaustive or limiting. Although methods and materials similar or equivalent to those described herein can be used in the practice or testing of the invention, suitable methods and materials are described herein.

The following terms, unless otherwise indicated, shall be understood to have the following meanings: the term “isolated molecule” as referring to a molecule (where the molecule is, for example, a protein, a polynucleotide, or an antibody) that by virtue of its origin or source of derivation (1) is not associated with naturally associated components that accompany it in its native state, (2) is substantially free of other molecules from the same source, e.g., species, cell from which it is expressed, library, etc., (3) is expressed by a cell from a different species, or (4) does not occur in nature. Thus, a molecule that is chemically synthesized, or expressed in a cellular system different from the system from which it naturally originates, will be “isolated” from its naturally associated components. A molecule also may be rendered substantially free of naturally associated components by isolation, using purification techniques well known in the art. Molecule purity or homogeneity may be assayed by a number of means well known in the art. For example, the purity of a polypeptide sample may be assayed using polyacrylamide gel electrophoresis and staining of the gel to visualize the polypeptide using techniques well known in the art. For certain purposes, higher resolution may be provided by using HPLC or other means well known in the art for purification.

As used herin, “variant”, “variant protein” or “protein variant” refers to a protein that differs from that of a parent protein by virtue of at least one amino acid modification. Protein variant may refer to the protein itself, a composition comprising the protein, or the amino sequence that encodes it. Preferably, the protein variant has at least one amino acid modification compared to the parent protein, e.g. from about one to about ten amino acid modifications, and preferably from about one to about five amino acid modifications compared to the parent. The protein variant sequence herein will preferably possess at least about 80% homology with a parent protein sequence, and most preferably at least about 90% homology, more preferably at least about 95% homology. Variant protein can refer to the variant protein itself, compositions comprising the protein variant, or the amino acid sequence that encodes it. Accordingly, by “IdeS variant” as used herein is meant a protein that differs from wildtype IdeS by virtue of at least one amino acid modification. Variants may comprise non-natural amino acids. Examples include U.S. Pat. No. 6,586,207; WO 98/48032; WO 03/073238; US2004-0214988A1; WO 05/35727A2; WO 05/74524A2; J. W. Chin et al., (2002), Journal of the American Chemical Society 124:9026-9027; J. W. Chin, & P. G. Schultz, (2002), ChemBioChem 11:1135-1137; J. W. Chin, et al., (2002), PICAS United States of America 99:11020-11024; and, L. Wang, & P. G. Schultz, (2002), Chem. 1-10.

As used herein, “protein” herein means at least two covalently attached amino acids, which includes proteins, polypeptides, oligopeptides and peptides. The peptidyl group may comprise naturally occurring amino acids and peptide bonds, or synthetic peptidomimetic structures, i.e. “analogs”, such as peptoids (see Simon et al., PNAS USA 89 (20): 9367 (1992)). The amino acids may either be naturally occurring or non-naturally occurring; as will be appreciated by those in the art. For example, homo-phenylalanine, citrulline, and noreleucine are considered amino acids for the purposes of the invention, and both D- and L-(R or S) configured amino acids may be utilized. The variants of the present invention may comprise modifications that include the use of unnatural amino acids incorporated using, for example, the technologies developed by Schultz and colleagues, including but not limited to methods described by Cropp & Shultz, 2004, Trends Genet. 20 (12): 625-30, Anderson et al., 2004, Proc Natl Acad Sci USA 101 (2): 7566-71, Zhang et al., 2003, 303 (5656): 371-3, and Chin et al., 2003, Science 301 (5635): 964-7. In addition, polypeptides may include synthetic derivatization of one or more side chains or termini, glycosylation, PEGylation, circular permutation, cyclization, linkers to other molecules, fusion to proteins or protein domains, and addition of peptide tags or labels.

As used herein, “wildtype” or “WT” refers to an amino acid sequence or a nucleotide sequence that is found in nature, including allelic variations. A wildtype protein has an amino acid sequence or a nucleotide sequence that has not been intentionally modified.

An “individual” or a “subject” is a mammal, more preferably, a human. Mammals also include, but are not limited to, farm animals (e.g., cows, pigs, horses, chickens, etc.), sport animals, pets, primates, horses, dogs, cats, mice and rats.

As used herein, an “effective dosage” or “effective amount” of drug, compound, or pharmaceutical composition is an amount sufficient to effect any one or more beneficial or desired results. In more specific aspects, an effective amount prevents, alleviates or ameliorates symptoms of disease, and/or prolongs the survival of the subject being treated. For prophylactic use, beneficial or desired results include eliminating or reducing the risk, lessening the severity, or delaying the outset of the disease, including biochemical, histological and/or behavioral symptoms of the disease, its complications and intermediate pathological phenotypes presenting during development of the disease. For therapeutic use, beneficial or desired results include clinical results such as reducing one or more symptoms of a disease, decreasing the dose of other medications required to treat the disease, enhancing the effect of another medication, and/or delaying the progression of the disease in patients. An effective dosage can be administered in one or more administrations. For purposes of this invention, an effective dosage of drug, compound, or pharmaceutical composition is an amount sufficient to accomplish prophylactic or therapeutic treatment either directly or indirectly. As is understood in the clinical context, an effective dosage of a drug, compound, or pharmaceutical composition may or may not be achieved in conjunction with another drug, compound, or pharmaceutical composition. Thus, an “effective dosage” may be considered in the context of administering one or more therapeutic agents, and a single agent may be considered to be given in an effective amount if, in conjunction with one or more other agents, a desirable result may be or is achieved.

The terms “nucleic acid” and “polynucleotide” are used interchangeably herein to refer to all forms of nucleic acid, oligonucleotides, including deoxyribonucleic acid (DNA) and ribonucleic acid (RNA). Nucleic acids include genomic DNA, cDNA and antisense DNA, and spliced or unspliced mRNA, rRNA tRNA and inhibitory DNA or RNA (RNAi, e.g., small or short hairpin (sh) RNA, microRNA (miRNA), small or short interfering (si) RNA, trans-splicing RNA, or antisense RNA).

A “heterologous” nucleic acid sequence refers to a polynucleotide inserted into a plasmid or vector for purposes of vector mediated transfer/delivery of the polynucleotide into a cell. Heterologous nucleic acid sequences are distinct from viral nucleic acid, i.e., are non-native with respect to viral nucleic acid. Once transferred/delivered into the cell, a heterologous nucleic acid sequence, contained within the vector, can be expressed (e.g., transcribed, and translated if appropriate). Alternatively, a transferred/delivered heterologous polynucleotide in a cell, contained within the vector, need not be expressed. Although the term “heterologous” is not always used herein in reference to nucleic acid sequences and polynucleotides, reference to a nucleic acid sequence or polynucleotide even in the absence of the modifier “heterologous” is intended to include heterologous nucleic acid sequences and polynucleotides in spite of the omission.

A “transgene” is used herein to conveniently refer to a nucleic acid that is intended or has been introduced into a cell or organism. Transgenes include any nucleic acid, such as a heterologous polynucleotide sequence or a heterologous nucleic acid encoding a protein or peptide. The term transgene and heterologous nucleic acid/polynucleotide sequences are used interchangeably herein.

A “host cell” includes an individual cell or cell culture that can be or has been a recipient for vector(s) for incorporation of polynucleotide inserts. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) of this invention.

As used herein, “vector” means a construct, which is capable of delivering, and, preferably, expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

The term “recombinant,” as a modifier of a viral vector, such as a recombinant AAV (rAAV) vector, as well as a modifier of sequences such as recombinant polynucleotides and polypeptides, means that compositions have been manipulated in a fashion that generally does not occur in nature. A particular example of a recombinant AAV vector would be where a nucleic acid that is not normally present in a wild-type AAV genome (heterologous polynucleotide) is inserted within a viral genome. An example of which would be where a nucleic acid (e.g., gene) encoding a therapeutic protein or polynucleotide sequence is cloned into a vector, with or without 5′, 3′ and/or intron regions that the gene is normally associated within the AAV genome. Although the term “recombinant” is not always used herein in reference to an AAV vector, as well as sequences such as polynucleotides, recombinant forms including AAV vectors, polynucleotides, etc., are expressly included notwithstanding any such omission.

A “rAAV vector,” for example, is derived from a wildtype genome of AAV by using molecular methods to remove all or a part of a wildtype AAV genome and replacing with a non-native (heterologous) nucleic acid, such as a nucleic acid encoding a therapeutic protein or polynucleotide sequence. Typically, for an rAAV vector one or both inverted terminal repeat (ITR) sequences of AAV genome are retained. A rAAV is distinguished from an AAV genome since all or a part of an AAV genome has been replaced with a non-native sequence with respect to the AAV genomic nucleic acid, such as with a heterologous nucleic acid encoding a therapeutic protein or polynucleotide sequence. Incorporation of a non-native (heterologous) sequence therefore defines an AAV as a″recombinant” AAV vector, which can be referred to as a “rAAV vector.”

A recombinant AAV vector sequence can be packaged and is referred to herein as a “particle” for subsequent infection (transduction) of a cell, ex vivo, in vitro or in vivo. Where a recombinant vector sequence is encapsidated or packaged into an AAV particle, the particle can also be referred to as a “rAAV,” “rAAV particle” and/or “rAAV virion.” Such rAAV, rAAV particles and rAAV virions include proteins that encapsidate or package a vector genome. In some embodiments such included proteins are capsid proteins.

A “vector genome,” which may be abbreviated as “vg”, refers to the portion of the recombinant plasmid sequence that is ultimately packaged or encapsidated to form an rAAV particle. In cases where recombinant plasmids are used to construct or manufacture recombinant AAV vectors, the AAV vector genome does not include the portion of the “plasmid” that does not correspond to the vector genome sequence of the recombinant plasmid. This non-vector genome portion of the recombinant plasmid is referred to as the “plasmid backbone,” which is important for cloning and amplification of the plasmid, a process that is needed for propagation and recombinant AAV vector production, but is not itself packaged or encapsidated into rAAV particles. Thus, a “vector genome” refers to the nucleic acid that is packaged or encapsidated by rAAV.

The term “PEG” refers to a polyethylene glycol, a linear, water-soluble polymer of ethylene PEG repeating units with two terminal hydroxyl groups. PEGs are classified by their molecular weights; for example, PEG 2000 has an average molecular weight of about 2,000 daltons, and PEG 5000 has an average molecular weight of about 5,000 daltons. PEGs are commercially available from Sigma Chemical Co. and other companies and include, for example, the following functional PEGs: monomethoxypolyethylene glycol (MePEG-OH), monomethoxypolyethylene glycol-succinate (MePEG-S), monomethoxypolyethylene glycol-succinimidyl succinate (MePEG-S-NHS), monomethoxypolyethylene glycol-amine (MePEG-NH2), monomethoxypolyethylene glycol-tresylate (MePEG-TRES), and monomethoxypolyethylene glycol-imidazolyl-carbonyl (MePEG-IM). In some embodiments, PEG may be a polyethylene glycol with an average molecular weight of about 550 to about 10,000 daltons and is optionally substituted by alkyl, alkoxy, acyl or aryl. In some embodiments, the PEG may be substituted with methyl at the terminal hydroxyl position. In some embodiments, the PEG may have an average molecular weight from about 750 to about 5,000 daltons, or from about 1,000 to about 5,000 daltons, or from about 1,500 to about 3,000 daltons or from about 2,000 daltons or of about 750 daltons. The PEG can be optionally substituted with alkyl, alkoxy, acyl or aryl. In some embodiments, the terminal hydroxyl group may be substituted with a methoxy or methyl group.

As used herein, the term “serotype” in reference to an AAV vector means a capsid that is serologically distinct from other AAV serotypes. Serologic distinctiveness is determined by lack of cross-reactivity between antibodies to one AAV as compared to another AAV. Cross-reactivity differences are usually due to differences in capsid protein sequences/antigenic determinants (e.g., due to VP1, VP2, and/or VP3 sequence differences of AAV serotypes). An antibody to one AAV may cross-react with one or more other AAV serotypes due to homology of capsid protein sequence. Under the traditional definition, a serotype means that the virus of interest has been tested against serum specific for all existing and characterized serotypes for neutralizing activity and no antibodies have been found that neutralize the virus of interest. As more naturally occurring virus isolates are discovered and/or capsid mutants generated, there may or may not be serological differences with any of the currently existing serotypes. Thus, in cases where the new virus (e.g., AAV) has no serological difference, this new virus (e.g., AAV) would be a subgroup or variant of the corresponding serotype. In many cases, serology testing for neutralizing activity has yet to be performed on mutant viruses with capsid sequence modifications to determine if they are of another serotype according to the traditional definition of serotype. Accordingly, for the sake of convenience and to avoid repetition, the term “serotype” broadly refers to both serologically distinct viruses (e.g., AAV) as well as viruses (e.g., AAV) that are not serologically distinct that may be within a subgroup or a variant of a given serotype.

As used herein, the term “effector function” in reference to an antibody means normal functional attributes of an antibody. Nonlimiting examples of antibody functional attributes include, for example, binding to an antigen; activation of the complement cascade (referred to as complement dependent cytotoxicity); binding to Fc receptor on effector cells, such as macrophages, monocytes, natural killer cells and eosinophils, to engage antibody-dependent cellular cytotoxicity (ADCC); and as a signal for ingestion of bound antigen/pathogen by immune cells such as phagocytes and dendritic cells. A reduction or inhibition of antibody effector function can therefore refer to any one or more of the foregoing nonlimiting functional attributes. Effector function assays are known in the art as well as described in WO2016012285, for example.

An “Fc receptor” refers to any Fc receptor. Particular non-limiting examples of Fc receptors include Fc gamma immunoglobulin receptors (FcγRs) which are present on cells. In humans, FcγR refers to one, some, or all of the family of Fc receptors comprising FcγRI (CD64), FcγRIIA (CD32A), FcγRIIB (CD32B), FcγRIIIA (CD16a) and FcγRIIIB (CD 16b). FcγR includes naturally occurring polymorphisms of FcγRI (CD64), FcγRIIA (CD32A), FcγRIIB (CD32B), FcγRIIIA (CD16a) and FcγRIIIB (CD 16b).

Reference to “about” a value or parameter herein includes (and describes) embodiments that are directed to that value or parameter per se. For example, description referring to “about X” includes description of “X.” Numeric ranges are inclusive of the numbers defining the range.

It is understood that wherever embodiments are described herein with the language “comprising,” otherwise analogous embodiments described in terms of “consisting of” and/or “consisting essentially of” are also provided. Throughout this specification and claims, the word “comprise,” or variations such as “comprises” or “comprising” will be understood to imply the inclusion of a stated integer or group of integers but not the exclusion of any other integer or group of integers.

Where aspects or embodiments of the invention are described in terms of a Markush group or other grouping of alternatives, the present invention encompasses not only the entire group listed as a whole, but each member of the group individually and all possible subgroups of the main group, but also the main group absent one or more of the group members. The present invention also envisages the explicit exclusion of one or more of any of the group members in the claimed invention.

All patents, patent applications, publications, and other references, GenBank citations and ATCC citations cited herein are incorporated by reference in their entirety. In case of conflict, the specification, including definitions, will control.

All of the features disclosed herein may be combined in any combination. Each feature disclosed in the specification may be replaced by an alternative feature serving a same, equivalent, or similar purpose. Thus, unless expressly stated otherwise, disclosed features are an example of a genus of equivalent or similar features

As used herein, the singular forms “a”, “and,” and “the” include plural referents unless the context clearly indicates otherwise. Thus, for example, reference to “a nucleic acid” includes a plurality of such nucleic acids, reference to “a vector” includes a plurality of such vectors, and reference to “a virus” or “particle” includes a plurality of such viruses/particles.

The term “about” as used herein refers to a value within 10% of the underlying parameter. For example, “about 1:10” means 1.1:10.1 or 0.9:9.9, and about 5 hours means 4.5 hours or 5.5 hours, etc. The term “about” at the beginning of a string of values modifies each of the values by 10%.

All numerical values or numerical ranges include integers within such ranges and fractions of the values or the integers within ranges unless the context clearly indicates otherwise. Thus, to illustrate, reference to reduction of 95% or more includes 95%, 96%, 97%, 98%, 99%, 100% etc., as well as 95.1%, 95.2%, 95.3%, 95.4%, 95.5%, etc., 96.1%, 96.2%, 96.3%, 96.4%, 96.5%, etc., and so forth. Thus, to also illustrate, reference to a numerical range, such as “1-4” includes 2, 3, as well as 1.1, 1.2, 1.3, 1.4, etc., and so forth. For example, “1 to 4 weeks” includes 7, 8, 9, 10, 11, 12, 13, 14, 15, 16, 17, 18, 19, 20, 21, 22, 23, 24, 25, 26, 27, or 28 days.

Further, reference to a numerical range, such as “0.01 to 10” includes 0.011, 0.012, 0.013, etc., as well as 9.5, 9.6, 9.7, 9.8, 9.9, etc., and so forth. For example, a dosage of about “0.01 mg/kg to about 10 mg/kg” body weight of a subject includes 0.011 mg/kg, 0.012 mg/kg, 0.013 mg/kg, 0.014 mg/kg, 0.015 mg/kg etc., as well as 9.5 mg/kg, 9.6 mg/kg, 9.7 mg/kg, 9.8 mg/kg, 9.9 mg/kg etc., and so forth.

Reference to an integer with more (greater) or less than includes any number greater or less than the reference number, respectively. Thus, for example, reference to more than 2 includes 3, 4, 5, 6, 7, 8, 9, 10, 11, 12, 13, 14, 15, etc., and so forth. For example, administration of a recombinant viral vector, IdeS variant “two or more” times includes 3, 4, 5, 6, 7, 8, 9, 10, 11, 12, 13, 14, 15, or more times. Further, reference to a numerical range, such as “1 to 90” includes 1.1, 1.2, 1.3, 1.4, 1.5, etc., as well as 81, 82, 83, 84, 85, etc., and so forth. For example, “between about 1 minute to about 90 days” includes 1.1 minutes, 1.2 minutes, 1.3 minutes, 1.4 minutes, 1.5 minutes, etc., as well as one day, 2 days, 3 days, 4 days, 5 days . . . 81 days, 82 days, 83 days, 84 days, 85 days, etc., and so forth.

Exemplary methods and materials are described herein, although methods and materials similar or equivalent to those described herein can also be used in the practice or testing of the present invention. The materials, methods, and examples are illustrative only and not intended to be limiting.

### IdeS Protein Variants

Provided herein are IdeS variants having improved potency and/or stability compared to wildtype IdeS. IdeS was discovered as a protein secreted in its mature form from Streptococcus pyogenes bacteria that specifically cleaves human immunoglobulin G (IgG, including its subclasses IgG1, IgG2, IgG3, and IgG4) in its hinge region, but not other classes of human immunoglobulin. The amino acid sequence of naturally occuring IdeS precursor corresponding to protein RefSeq WP_010922160.1 is provided in Table 1 as SEQ ID NO:1. That protein includes a 29 amino acid long secretion signal peptide, the sequence of which is provided as SEQ ID NO:83, and a 310 amino acid long mature polypeptide, the sequence of which is provided as SEQ ID NO:84. Further details regarding the structure of IdeS can be found, for example, in von Pawel-Rammingen U, Johansson BP, Björck L. IdeS, a novel streptococcal cysteine proteinase with unique specificity for immunoglobulin G. EMBO J. 2002 Apr. 2;21 (7): 1607-15. doi: 10.1093/emboj/21.7.1607; Wenig K, et al. Structure of the streptococcal endopeptidase IdeS, a cysteine proteinase with strict specificity for IgG. Proc Natl Acad Sci USA. 2004 Dec. 14;101 (50): 17371-6. doi: 10.1073/pnas.0407965101.

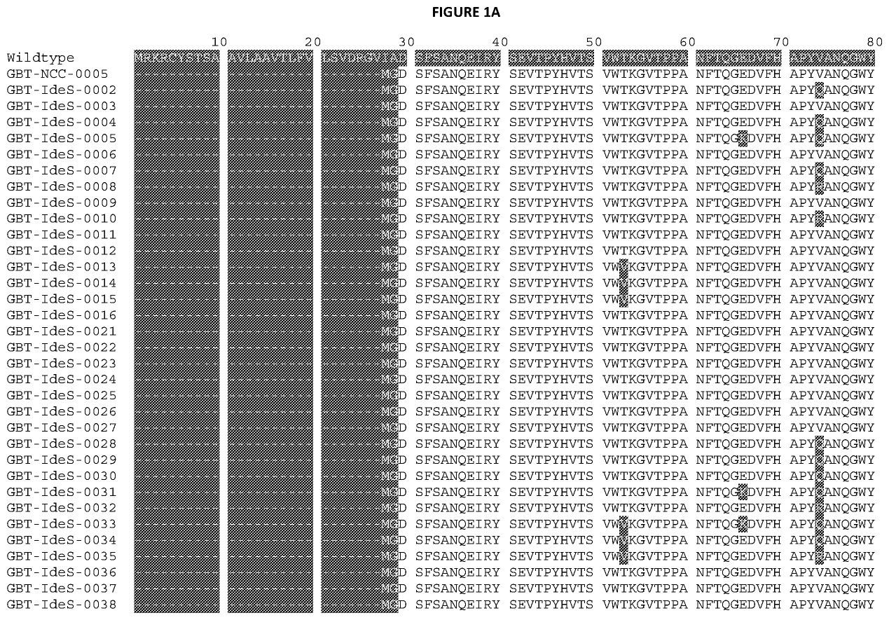

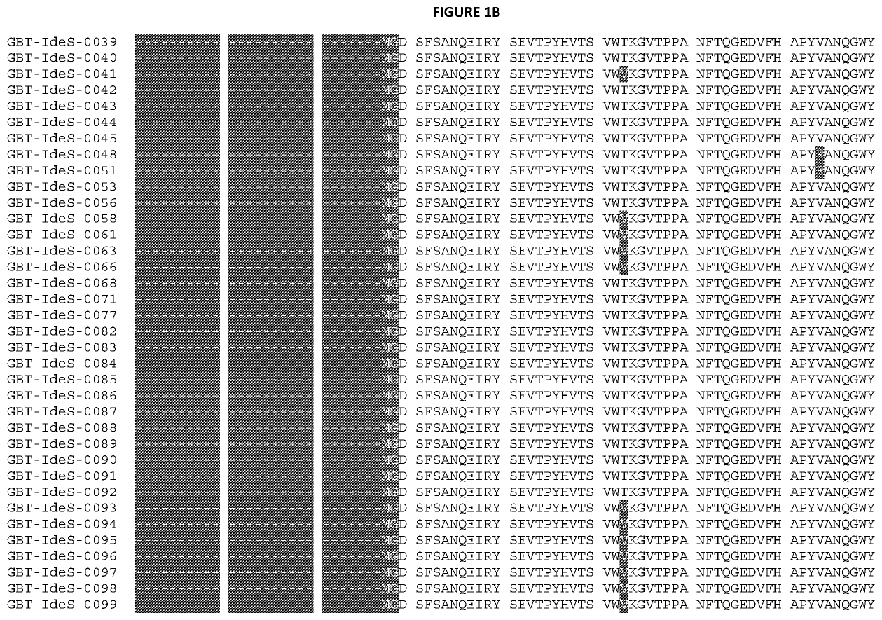

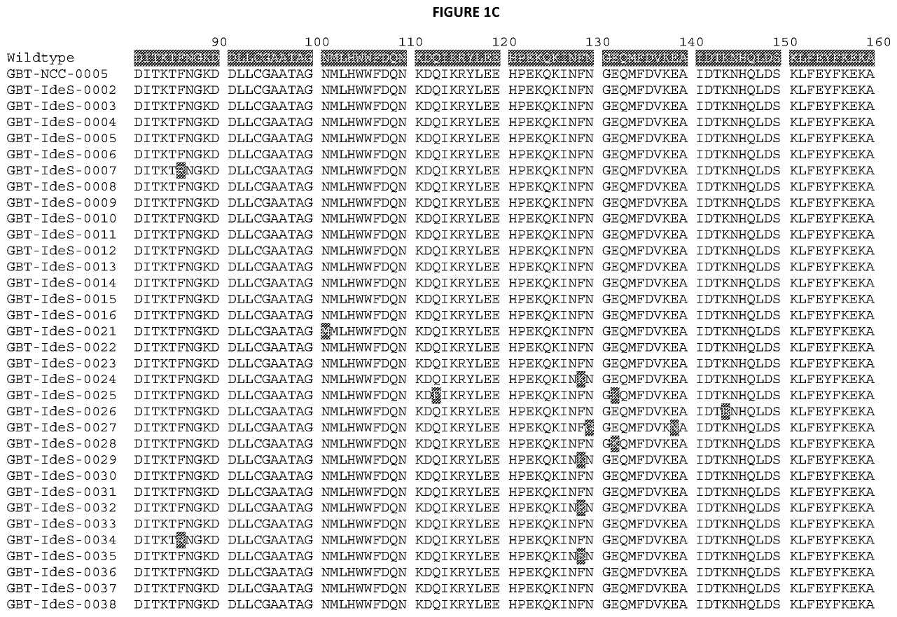

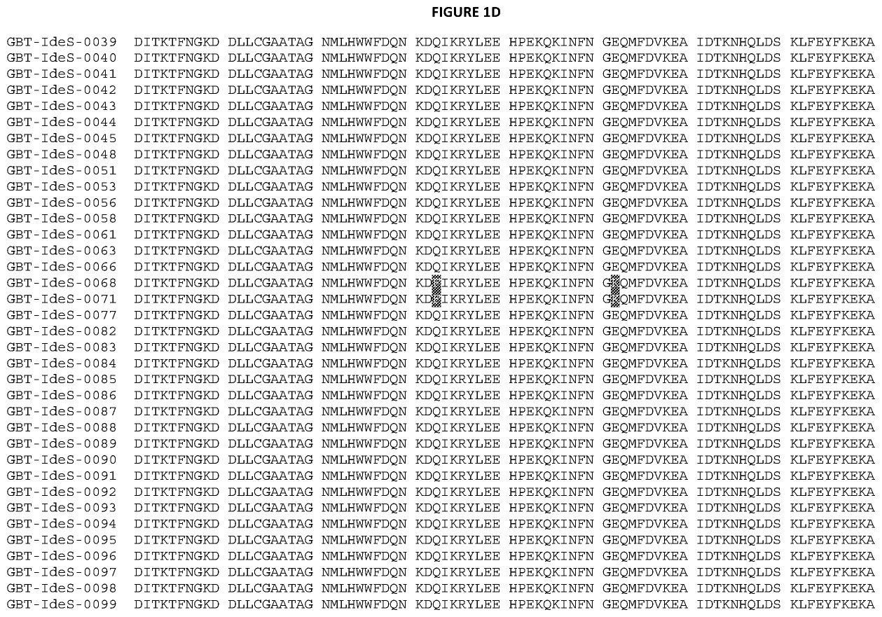

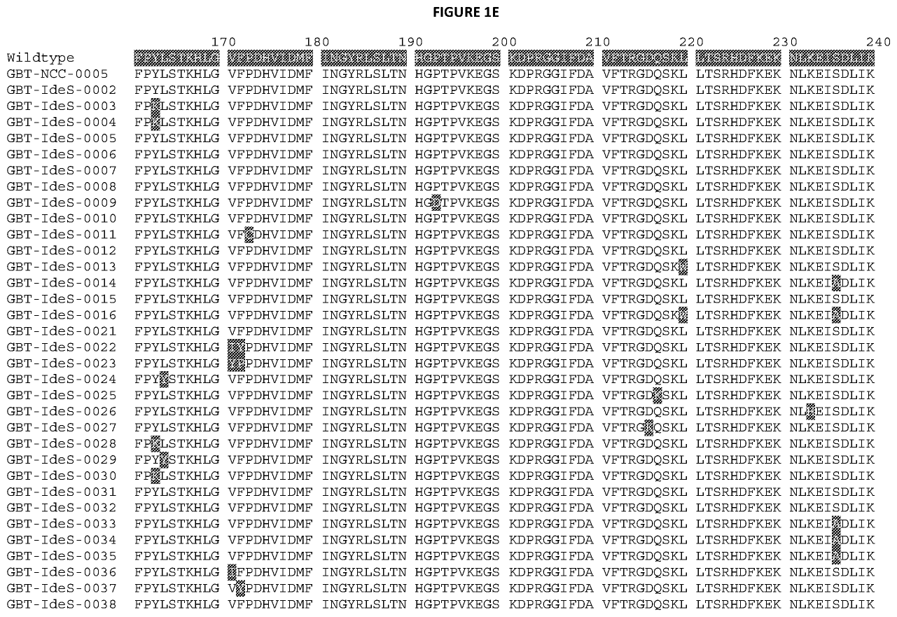

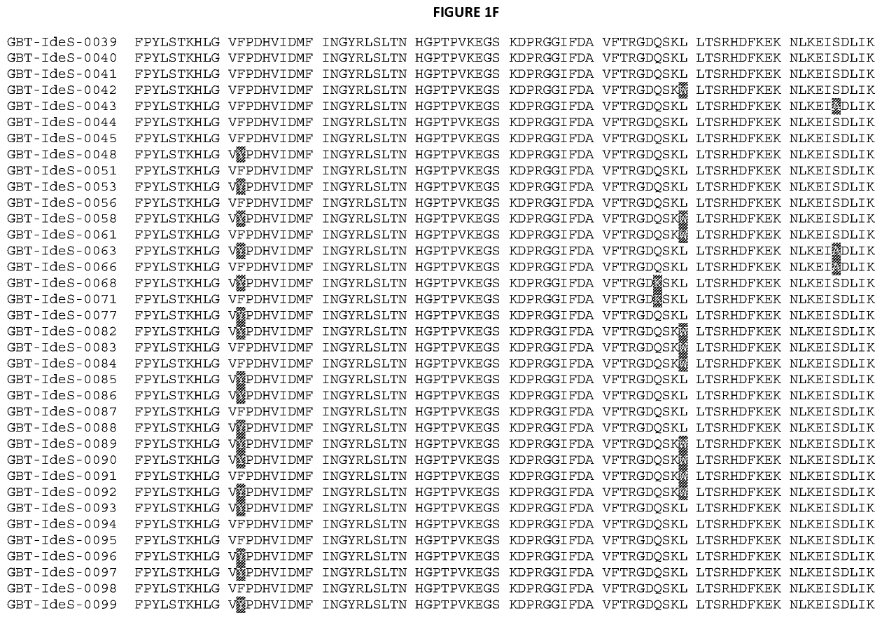

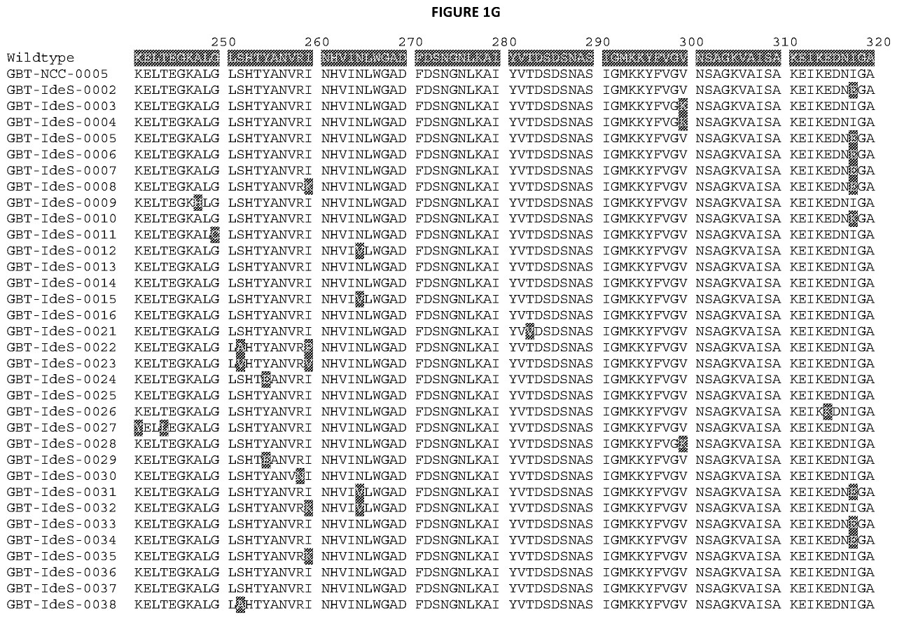

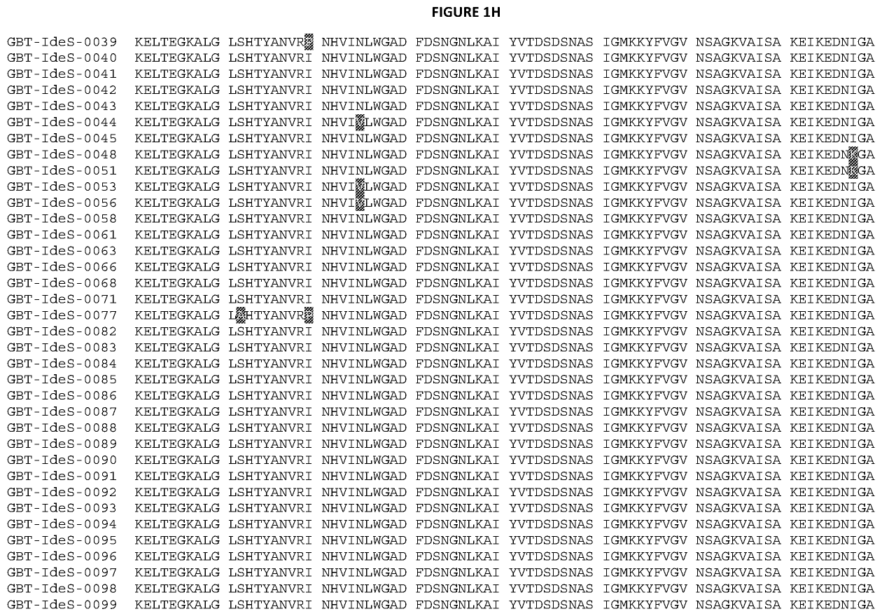

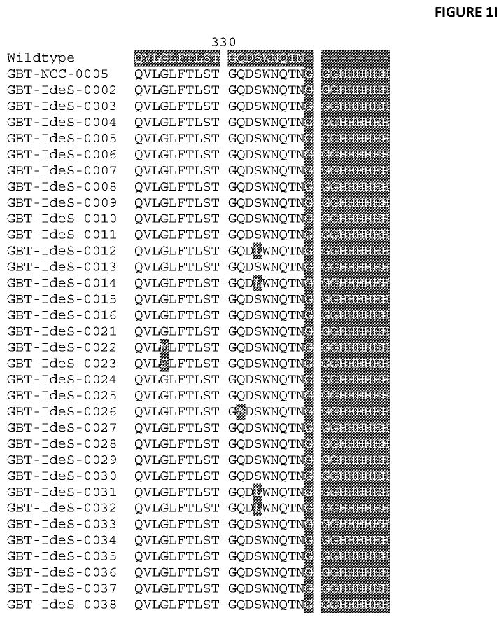

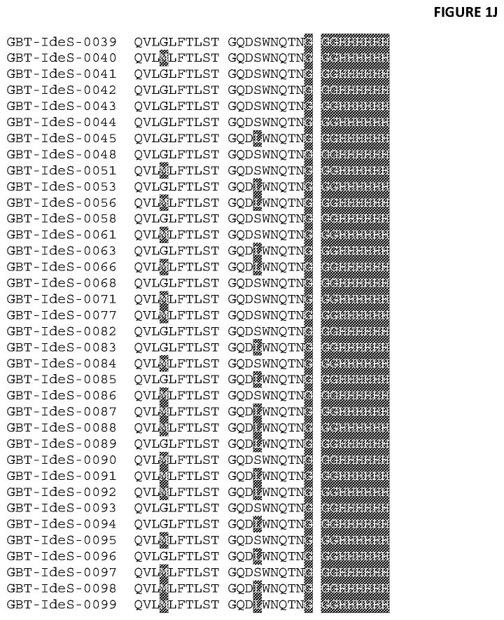

Based on a rational protein design approach, the disclosure surprisingly provides certain substitution variants of wildtype IdeS that are more thermostable and/or are more potent in their ability to cleave IgG molecules as compared to the wildtype IdeS protein sequence from which they were derived. Names and amino acid sequences of novel IdeS variants are disclosed in FIGS. 1A-1J aligned with the reference sequence for wildtype Ides protein (RefSeq WP_010922160.1). Wildtype IdeS is illustrated in its precursor form (SEQ ID NO: 1) including the naturally occurring secretion signal peptide (SEQ ID NO:83) positioned at the amino-terminus of the mature polypeptide (SEQ ID NO:84), whereas the IdeS variants instead start with the dipeptide Met-Gly and end with a His tag (SEQ ID NO:85) not present in wildtype, facilitating small scale purification. Amino acid differences between the variants and wildtype IdeS are highlighted. The amino acid sequences of wildtype IdeS and the named variants depicted in FIGS. 1A-1J are further identified by SEQ ID NO as set forth in Table 1, below, along with the other amino acid sequences disclosed herein. Although the variants depicted in FIGS. 1A-1J possess different amino- and carboxy-termini compared to wildtype IdeS, these differences should be considered exemplary and not limiting. Thus, for example, any variant could, rather than starting with Met-Gly, start with the naturally occurring Ides secretion signal peptide, or some other sequence of amino acids at its amino-terminus. Furthermore, any variant could, rather than end with a His tag as illustrated, end with no tag, similar to wildtype, or end with a different tag or some other sequence of amino acids at its carboxy-terminus. Thus, for example, in some embodiments, a variant of the disclosure can comprise amino acid numbers 1 through 312 of any one of SEQ ID NOs 2 through 71, or can comprise amino acid numbers 2 through 312 of any one of SEQ ID NOs 2 through 71, or can comprise amino acid numbers 3 through 312 of any one of SEQ ID NOs 3 through 71, or any other range of amino acids comprised by SEQ ID NOs 2 through 71, or subsequences thereof.

TABLE 1

| | Amino Acid Sequence |
|---|---|
| Protein Name | Disclosure by SEQ ID NO |
| Wildtype (WT) IdeS | 1 |
| RefSeq WP_010922160.1 | |
| GBT-NCC-0005 | 2 |
| GBT-IdeS-0002 | 3 |
| GBT-IdeS-0003 | 4 |
| GBT-IdeS-0004 | 5 |
| GBT-IdeS-0005 | 6 |
| GBT-IdeS-0006 | 7 |
| GBT-IdeS-0007 | 8 |
| GBT-IdeS-0008 | 9 |
| GBT-IdeS-0009 | 10 |
| GBT-IdeS-0010 | 11 |
| GBT-IdeS-0011 | 12 |
| GBT-IdeS-0012 | 13 |
| GBT-IdeS-0013 | 14 |
| GBT-IdeS-0014 | 15 |
| GBT-IdeS-0015 | 16 |
| GBT-IdeS-0016 | 17 |
| GBT-IdeS-0021 | 18 |
| GBT-IdeS-0022 | 19 |
| GBT-IdeS-0023 | 20 |
| GBT-IdeS-0024 | 21 |
| GBT-IdeS-0025 | 22 |
| GBT-IdeS-0026 | 23 |
| GBT-IdeS-0027 | 24 |
| GBT-IdeS-0028 | 25 |
| GBT-IdeS-0029 | 26 |
| GBT-IdeS-0030 | 27 |
| GBT-IdeS-0031 | 28 |
| GBT-IdeS-0032 | 29 |
| GBT-IdeS-0033 | 30 |
| GBT-IdeS-0034 | 31 |
| GBT-IdeS-0035 | 32 |
| GBT-IdeS-0036 | 33 |
| GBT-IdeS-0037 | 34 |
| GBT-IdeS-0038 | 35 |
| GBT-IdeS-0039 | 36 |
| GBT-IdeS-0040 | 37 |
| GBT-IdeS-0041 | 38 |
| GBT-IdeS-0042 | 39 |
| GBT-IdeS-0043 | 40 |
| GBT-IdeS-0044 | 41 |
| GBT-IdeS-0045 | 42 |
| GBT-IdeS-0048 | 43 |
| GBT-IdeS-0051 | 44 |
| GBT-IdeS-0053 | 45 |
| GBT-IdeS-0056 | 46 |
| GBT-IdeS-0058 | 47 |
| GBT-IdeS-0061 | 48 |
| GBT-IdeS-0063 | 49 |
| GBT-IdeS-0066 | 50 |
| GBT-IdeS-0068 | 51 |
| GBT-IdeS-0071 | 52 |
| GBT-IdeS-0077 | 53 |
| GBT-IdeS-0082 | 54 |
| GBT-IdeS-0083 | 55 |
| GBT-IdeS-0084 | 56 |
| GBT-IdeS-0085 | 57 |
| GBT-IdeS-0086 | 58 |
| GBT-IdeS-0087 | 59 |
| GBT-IdeS-0088 | 60 |
| GBT-IdeS-0089 | 61 |
| GBT-IdeS-0090 | 62 |
| GBT-IdeS-0091 | 63 |
| GBT-IdeS-0092 | 64 |
| GBT-IdeS-0093 | 65 |
| GBT-IdeS-0094 | 66 |
| GBT-IdeS-0095 | 67 |
| GBT-IdeS-0096 | 68 |
| GBT-IdeS-0097 | 69 |
| GBT-IdeS-0098 | 70 |
| GBT-IdeS-0099 | 71 |
| PF-07826653 | 72 |
| PF-07826653 | 73 |
| (not including N- | |
| terminal Met) | |
| PF-07899856 | 74 |
| PF-07899856 | 75 |
| (not including N- | |
| terminal Met) | |
| PF-07899856 | 76 |
| (not including N- | |
| terminal Met Gly) | |
| PF-07862203 | 77 |
| PF-07862203 | 78 |
| (not including N- | |
| terminal Met) | |
| PF-07862203 | 79 |
| (not including N- | |
| terminal Met Gly) | |
| GBT-IdeS-0100 | 80 |
| GBT-IdeS-0100 | 81 |
| (not including N- | |
| terminal Met) | |
| GBT-IdeS-0100 | 82 |
| (not including N- | |
| terminal Met Gly) | |
| Wildtype IdeS | 83 |
| secretion signal | |
| Wildtype IdeS | 84 |
| mature protein | |
| His tag | 85 |

In some embodiments, the amino acid sequence of IdeS variant proteins of the disclosure comprise, consist, or consist essentially of the amino acid sequence of the mature wildtype IdeS protein (SEQ ID NO:84) in which one or more amino acids is substituted with a different amino acid, and/or in which one or more amino acids is inserted or deleted, and/or to which one or more amino acids is added at its amino terminus, and/or to which one or more amino acids is added at its carboxy terminus.

In some embodiments, the amino acid sequence of an IdeS variant protein of the disclosure comprises, consists, or consists essentially of (a) amino acid numbers 1-312 of any one of SEQ ID NOs: 2-71, or amino acid numbers 2-312 of any one of SEQ ID NOs: 2-71, or amino acid numbers 3-312 of any one of SEQ ID NOs: 3-71; or (b) a fragment of (a) having lg endopeptidase activity; or (c) a variant of (a) having at least 50% identity to amino acid numbers 1-312 of any one of SEQ ID NOs: 2-71, or amino acid numbers 2-312 of any one of SEQ ID NOs: 2-71, or amino acid numbers 3-312 of any one of SEQ ID NOs: 3-71 and having lg endopeptidase activity; or (d) a variant of (b) having at least 50% identity to the corresponding portion of amino acid numbers 1-312 of any one of SEQ ID NOs: 2-71, or amino acid numbers 2-312 of any one of SEQ ID NOs: 2-71, or amino acid numbers 3-312 of any one of SEQ ID NOS: 3-71 and having Ig endopeptidase activity.

In some embodiments, the IdeS variant protein has at least about 60% or more identity (e.g., 60-70%, 70-80%, 80-90%, or 90-100% identity) to amino acid numbers 1-312 of any one of SEQ ID NOs: 2-71, or amino acid numbers 2-312 of any one of SEQ ID NOs: 2-71, or amino acid numbers 3-312 of any one of SEQ ID NOs: 3-71, or a fragment thereof having lg endopeptidase activity. In some embodiments, the IdeS variant protein has at least about 70% or more identity (e.g., 70-80%, 80-90%, or 90-100% identity) to amino acid numbers 1-312 of any one of SEQ ID NOs: 2-71, or amino acid numbers 2-312 of any one of SEQ ID NOs: 2-71, or amino acid numbers 3-312 of any one of SEQ ID NOs: 3-71, or a fragment thereof having lg endopeptidase activity. In some embodiments, the IdeS variant protein has at least about 80% or more identity (e.g., 80-90%, or 90-100% identity) to amino acid numbers 1-312 of any one of SEQ ID NOs: 2-71, or amino acid numbers 2-312 of any one of SEQ ID NOs: 2-71, or amino acid numbers 3-312 of any one of SEQ ID NOs: 3-71, or a fragment thereof having lg endopeptidase activity. In some embodiments, the IdeS variant protein has at least about 90% or more identity (e.g., 90-100% identity) to amino acid numbers 1-312 of any one of SEQ ID NOs: 2-71, or amino acid numbers 2-312 of any one of SEQ ID NOs: 2-71, or amino acid numbers 3-312 of any one of SEQ ID NOs: 3-71, or a fragment thereof having Ig endopeptidase activity. In some embodiments, the IdeS variant protein has at least about 95% or more identity (e.g., 95-100% identity) to amino acid numbers 1-312 of any one of SEQ ID NOs: 2-71, or amino acid numbers 2-312 of any one of SEQ ID NOs: 2-71, or amino acid numbers 3-312 of any one of SEQ ID NOs: 3-71, or a fragment thereof having Ig endopeptidase activity. In some embodiments, the IdeS variant protein has at least about 96% or more identity (e.g., 96-100% identity) to amino acid numbers 1-312 of any one of SEQ ID NOs: 2-71, or amino acid numbers 2-312 of any one of SEQ ID NOs: 2-71, or amino acid numbers 3-312 of any one of SEQ ID NOS: 3-71, or a fragment thereof having Ig endopeptidase activity. In some embodiments, the IdeS variant protein has at least about 97% or more identity (e.g., 97-100% identity) to amino acid numbers 1-312 of any one of SEQ ID NOs: 2-71, or amino acid numbers 2-312 of any one of SEQ ID NOs: 2-71, or amino acid numbers 3-312 of any one of SEQ ID NOS: 3-71, or a fragment thereof having Ig endopeptidase activity. In some embodiments, the IdeS variant protein has at least about 98% or more identity (e.g., 98-100% identity) to amino acid numbers 1-312 of any one of SEQ ID NOs: 2-71, or amino acid numbers 2-312 of any one of SEQ ID NOs: 2-71, or amino acid numbers 3-312 of any one of SEQ ID NOs: 3-71, or a fragment thereof having Ig endopeptidase activity. In some embodiments, the IdeS variant protein has at least about 99% or more identity (e.g., 99-100% identity) to amino acid numbers 1-312 of any one of SEQ ID NOs: 2-71, or amino acid numbers 2-312 of any one of SEQ ID NOs: 2-71, or amino acid numbers 3-312 of any one of SEQ ID NOs: 3-71, or a fragment thereof having lg endopeptidase activity.

In some embodiments, the amino acid sequence of an IdeS variant protein of the disclosure comprises amino acid numbers 1-312 of any one of SEQ ID NOs: 2-71, or amino acid numbers 2-312 of any one of SEQ ID NOs: 2-71, or amino acid numbers 3-312 of any one of SEQ ID NOs: 3-71. In some embodiments, the amino acid sequence of an IdeS variant protein of the disclosure consists essentially of amino acid numbers 1-312 of any one of SEQ ID NOs: 2-71, or amino acid numbers 2-312 of any one of SEQ ID NOs: 2-71, or amino acid numbers 3-312 of any one of SEQ ID NOs: 3-71. In some embodiments, the amino acid sequence of an IdeS variant protein of the disclosure consists of amino acid numbers 1-312 of any one of SEQ ID NOs: 2-71, or amino acid numbers 2-312 of any one of SEQ ID NOs: 2-71, or amino acid numbers 3-312 of any one of SEQ ID NOs: 3-71.

In some embodiments, IdeS variant proteins of the disclosure comprise, consist, or consist essentially of an amino acid sequence which corresponds to the mature wildtype Ides protein (SEQ ID NO:84) in that they are devoid of a secretion signal peptide sequence positioned at the amino-terminus of the protein, or any other type of extraneous peptide sequences positioned at the amino- or carboxy-termini of such proteins. Non-limiting examples of such embodiments include IdeS variant proteins the amino acid sequences of which are provided in SEQ ID NOs: 76, 79, and 82. Additional examples include IdeS protein variants comprising, consisting of, or consisting essentially of amino acid numbers 3-312 of any one of SEQ ID NOs: 3-71.

In some embodiments, IdeS variant proteins of the disclosure comprise a prokaryotic secretion signal peptide sequence positioned at the amino-terminus of the protein, which facilitates export of the IdeS protein from a bacterial cell after its expression. Such signal peptide sequence may be the naturally occurring signal peptide sequence of Ides, as provided by SEQ ID NO:83, or a signal peptide sequence from another secreted protein of S. pyogenes. Thus, for example, in some embodiments, the IdeS protein variants as provided in SEQ ID NOs: 76, 79, and 82 may each further comprise the amino acid sequence of SEQ ID NO: 83 at the amino-terminus of each such protein. Additional examples include IdeS protein variants comprising amino acid numbers 3-312 of any one of SEQ ID NOs: 3-71 each further comprising the amino acid sequence of SEQ ID NO:83 at the amino-terminus of each such protein. In some other embodiments, IdeS protein variants of the disclosure may comprise a signal peptide from a secreted protein of a different species of bacteria than S. pyogenes, such as E. coli, or another species. Further information about bacterial signal peptides may be found, for example, in Freudl, R. Signal peptides for recombinant protein secretion in bacterial expression systems. Microb Cell Fact 17, 52 (2018). doi: 10.1186/s12934-018-0901-3; and Kaushik S, He H and Dalbey RE (2022) Bacterial Signal Peptides-Navigating the Journey of Proteins. Front. Physiol. 13:933153. doi: 10.3389/fphys.2022.933153; Green ER, Mecsas J. Bacterial Secretion Systems: Overview. An Microbiol Spectr. 2016 February;4 (1): 10.1128/microbiolspec.VMBF-0012-2015. doi: 10.1128/microbiolspec.VMBF-0012-2015; and Kleiner-Grote GRM, Risse JM, Friehs K. Secretion of recombinant proteins from E. coli. Eng Life Sci. 2018 Apr. 14;18 (8): 532-550. doi: 10.1002/elsc.201700200.

In some embodiments, such as where an IdeS variant protein is intended to be expressed in a eukaryotic cell, IdeS variant proteins of the disclosure can comprise a eukaryotic secretion signal peptide sequence corresponding to the type of cell in which the variant protein would be expressed. Examples of eukaryotic cells in which IdeS variant proteins may be expressed include yeast cells, plant cells, insect cells, or mammalian cells, as well as other types.

In some embodiments, IdeS variant proteins of the disclosure comprise a short peptide sequence positioned at the amino-terminus of the protein beginning with methionine, which is encoded by DNA or RNA sequence sufficient to support translation of the IdeS variant, but that does not necessarily target the expressed protein for secretion from the cell in which it is expressed. For example, in some embodiments, an IdeS variant protein (or wildtype IdeS) may start with the dipeptide sequence Met-Gly (or MG in single letter code) which is then followed by the mature protein amino acid sequence. In such embodiments, where an IdeS variant protein is not translated with a secretion signal peptide sequence (for example, where such proteins start with MG or some other translatable sequence), the protein may collect intracellularly as it is expressed, and can be released by lysing the cells and then purifying the proteins so released using methods familiar to those of ordinary skill. Thus, for example, in some embodiments, the IdeS protein variants as provided in SEQ ID NOS: 76, 79, and 82 may each further comprise the dipeptide amino acid sequence Met-Gly (MG) at the amino-terminus of each such protein. Additional examples include the IdeS protein variants as provided in SEQ ID NOs: 2-71, each of which start with Met-Gly (MG), and which, in some other embodiments, may each be provided without the last nine amino acids appearing at the respective carboxy-termini of SEQ ID NOs: 2-71, corresponding to the His tag of SEQ ID NO:85. Such embodiments may also be described as IdeS protein variants comprising, consisting, or consisting essentially of amino acid numbers 1-312 of any one of SEQ ID NOs: 2-71.

In some embodiments, the amino-terminal methionine (M) may be removed in the course of expression or subsequent purification, such as through the action of endogenous aminopeptidases, or other mechanisms, such that the predominant species of IdeS variant protein lacks a methionine (Met, M) at its amino-terminus. Present at the amino-terminus would instead be the subsequent amino acid positioned immediately after the methionine before its removal. Thus, for example, in some embodiments, the IdeS protein variants as provided in SEQ ID NOs: 76, 79, 82, and 84 may each further comprise the single amino acid Gly (G) at the amino-terminus of each such protein. Additional examples include IdeS protein variants comprising amino acid numbers 3-312 of any one of SEQ ID NOs: 2-71 each further comprising the single amino acid Gly (G) at the amino-terminus of each such protein. Such embodiments may also be described as IdeS protein variants comprising, consisting, or consisting essentially of amino acid numbers 2-312 of any one of SEQ ID NOs: 2-71. Yet further examples include the IdeS protein variants as provided in SEQ ID NOs: 73, 75, 78, and 81, each of which starts with Gly (G).

In some embodiments, an IdeS protein variant of the disclosure comprises a short sequence of amino acids positioned at the amino- or carboxy-terminus of the protein which confers some desired function, such as affinity capture using an antibody metal ion resin. Commonly used peptide and protein tags include those known as CBP, FLAG, GST, HA, HBH, MBP, Myc, Poly-His, S-tag, SUMO, TAP, TRX, and V5, others being possible. Such tags may also be provided with protease cleavage sites so as to allow post-translational removal of the tags when desired, non-limiting examples including those known as TEV, thrombin, and PreScission sites, others being possible. Tags and other functional peptide and protein moieties that may be fused to an IdeS protein variant of the disclosure are described in Kimple M E, Brill A L, Pasker R L. Overview of affinity tags for protein purification. Curr Protoc Protein Sci. 2013 Sep. 24; 73:9.9.1-9.9.23. doi: 10.1002/0471140864.ps0909s73.

Modification of polypeptides is routine practice in the art and need not be described in detail herein. Examples of modified polypeptides include polypeptides with conservative substitutions of amino acid residues, one or more deletions or additions of amino acids which do not significantly deleteriously change the functional activity, or which mature (enhance) the affinity of the polypeptide for its ligand, or use of chemical analogs.

Amino acid sequence insertions include amino- and/or carboxy-terminal fusions ranging in length from one residue to polypeptides containing a hundred or more residues, as well as intrasequence insertions of single or multiple amino acid residues. Examples of terminal insertions include an IdeS protein with an N-terminal methionyl residue or the IdeS protein fused to an epitope tag. Other insertional variants of the IdeS protein include the fusion to the N- or C-terminus of the IdeS protein of an enzyme or a polypeptide which increases the half-life of the IdeS protein in the blood circulation.

Substitution variants have at least one amino acid residue in the IdeS protein removed and a different residue inserted in its place. Conservative substitutions are shown in Table 2 under the heading of “conservative substitutions.” If such substitutions result in a change in biological activity, then more substantial changes, denominated “exemplary substitutions” in Table 2, or as further described below in reference to amino acid classes, may be introduced and the products screened.

TABLE 2

| Original | Conservative | Exemplary |
|---|---|---|
| Residue | Substitutions | Substitutions |
| Ala (A) | Val | Val; Leu; Ile |
| Arg (R) | Lys | Lys; Gln; Asn |
| Asn (N) | Gln | Gln; His; Asp, Lys; Arg |
| Asp (D) | Glu | Glu; Asn |
| Cys (C) | Ser | Ser; Ala |
| Gln (Q) | Asn | Asn; Glu |
| Glu (E) | Asp | Asp; Gln |
| Gly (G) | Ala | Ala |
| His (H) | Arg | Asn; Gln; Lys; Arg |
| Ile (I) | Leu | Leu; Val; Met; Ala; Phe; |
| | | Norleucine |
| Leu (L) | Ile | Norleucine; Ile; Val; Met; Ala; |
| | | Phe |
| Lys (K) | Arg | Arg; Gln; Asn |
| Met (M) | Leu | Leu; Phe; Ile |
| Phe (F) | Tyr | Leu; Val; Ile; Ala; Tyr |
| Pro (P) | Ala | Ala |
| Ser (S) | Thr | Thr |
| Thr (T) | Ser | Ser |
| Trp (W) | Tyr | Tyr; Phe |
| Tyr (Y) | Phe | Trp; Phe; Thr; Ser |
| Val (V) | Leu | Ile; Leu; Met; Phe; Ala; |
| | | Norleucine |

Substantial modifications in the biological properties of the IdeS protein may be accomplished by selecting substitutions that differ significantly in their effect on maintaining (a) the structure of the polypeptide backbone in the area of the substitution, for example, as a E-sheet or helical conformation, (b) the charge or hydrophobicity of the molecule at the target site, or (c) the bulk of the side chain. Naturally occurring residues are divided into groups based on common side-chain properties:
(1) Non-polar: Norleucine, Met, Ala, Val, Leu, Ile;
(2) Polar without charge: Cys, Ser, Thr, Asn, Gln;
(3) Acidic (negatively charged): Asp, Glu;
(4) Basic (positively charged): Lys, Arg;
(5) Residues that influence chain orientation: Gly, Pro; and
(6) Aromatic: Trp, Tyr, Phe, His.

Non-conservative substitutions are made by exchanging a member of one of these classes for another class.

The IdeS variant of interest may be sequenced and the polynucleotide sequence may then be cloned into a vector for expression or propagation. The sequence encoding IdeS variant of interest may be maintained in vector in a host cell and the host cell can then be expanded and frozen for future use.

In some embodiments, IdeS variants of the disclosure are more thermostable as compared to wildtype IdeS. Improved thermostability is desirable, in some embodiments, because it can improve manufacturability, formulation, long term storage, deliver to patients, and efficacy. Numerous methods for quantifying protein stability are known in the art and can be used to measure thermostability of IdeS variant proteins in comparison to wildtype IdeS, examples including circular dichroism (CD), dynamic and static light scattering (DLS and SLS), size exclusion chromatography with multi-angle light scattering (SEC-MALS), Fourier transform infrared spectroscopy (FTIR), analytical ultrafiltration (AUC), size exclusion chromatography (SEC), differential scanning fluorescence (DSF), intrinsic fluorescence (IF), and differential scanning calorimetry (DSC).

In some embodiments, thermostability of IdeS variants is quantified using DSC, which generates two values that can be used to conveniently express thermostability. Tonset is the temperature at which a pure protein begins to denature, and TM is the thermal transition temperature at which 50% of the protein is in its native conformation and 50% is denatured.

In some embodiments, an IdeS variant of the disclosure has a Tₒₙₛₑₜ value that is at least or about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4.0° C. greater compared to wildtype IdeS protein, or more, or any value between or range encompassing any of the foregoing specifically enumerated values. In some embodiments, an IdeS variant of the disclosure has a Tₒₙₛₑₜ value that is at least or about 44.0, 44.1, 44.2, 44.3, 44.4, 44.5, 44.6, 44.7, 44.8, 44.9, 45.0, 45.1, 45.2, 45.3, 45.4, 45.5, 45.6, 45.7, 45.8, 45.9, 46.0, 46.1, 46.2, 46.3, 46.4, 46.5, 46.6, 46.7, 46.8, 46.9, 47.0, 47.1, 47.2, 47.3, 47.4, 47.5, 47.6, 47.7, 47.8, 47.9, 48.0, 48.1, 48.2, 48.3, 48.4, 48.5, 48.6, 48.7, 48.8, 48.9, 49.0, 49.1, 49.2, 49.3, 49.4, 49.5, 49.6, 49.7, 49.8, 49.9, or 50.0° C., or more, or any value between or range encompassing any of the foregoing specifically enumerated values.

In some embodiments, an IdeS variant of the disclosure has a TM value that is at least or about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4.0° C. greater compared to wildtype IdeS protein, or more, or any value between or range encompassing any of the foregoing specifically enumerated values. In some embodiments, an IdeS variant of the disclosure has a TM value that is at least or about 51.0, 51.1, 51.2, 51.3, 51.4, 51.5, 51.6, 51.7, 51.8, 51.9, 52.0, 52.1, 52.2, 52.3, 52.4, 52.5, 52.6, 52.7, 52.8, 52.9, 53.0, 53.1, 53.2, 53.3, 53.4, 53.5, 53.6, 53.7, 53.8, 53.9, 54.0, 54.1, 54.2, 54.3, 54.4, 54.5, 54.6, 54.7, 54.8, 54.9, 55.0, 55.1, 55.2, 55.3, 55.4, 55.5, 55.6, 55.7, 55.8, 55.9, 56.0, 56.1, 56.2, 56.3, 56.4, 56.5, 56.6, 56.7, 56.8, 56.9, or 57.0° C., or more, or any value between or range encompassing any of the foregoing specifically enumerated values.

In some embodiments, IdeS variants of the disclosure are more potent as compared to wildtype IdeS. Improved potency is desirable, in some embodiments, because it permits the same amount of target IgG to be cleaved with a lower mass, concentration or dose of an IdeS variant as compared to wildtype IdeS. Several methods for quantifying IdeS enzymatic potency have been described, and can be used to measure potency of IdeS variant proteins in comparison to wildtype Ides, for example, ELISA-based methods, visualization of the products of IdeS digestion of IgG, or mass spectroscopic methods, such as that described in Hess, J L, et al., Immunoglobulin cleavage by the streptococcal cysteine protease IdeS can be detected using protein G capture and mass spectrometry, J. Microbiol. Meths., 70 (2): 284-291 (2007) (doi.org/10.1016/j.mimet.2007.04.017).

In some embodiments, potency of IdeS variants of the disclosure can be measured using an ELISA based sandwich assay. In one version of this assay, human IgG is immobilized to an ELISA plate through a capture antibody (such as mouse anti-human) specific for the human IgG F(ab) region. IdeS protein (variant or wildtype) is then added to the wells of the plate in varying concentrations and incubated for a predetermined time, which acts to cleave the human IgG below the hinge in a sequential process, producing single cleaved IgG (scIgG), and then an F(ab′)2 fragment and two Fc monomers. Vindebro, R, et al., Rapid IgG heavy chain cleavage by the streptococcal IgG endopeptidase IdeS is mediated by IdeS monomers and is not due to enzyme dimerization, FEBS Letters 587 (12): 1818-1822 (2013) (doi.org/10.1016/j.febslet.2013.04.039). The amount of intact human IgG versus partially or fully cleaved IgG can then be measured using a detector antibody specific for the dimeric human IgG Fc domain. Results can be expressed as in IC50 value, in which lower numbers are indictative of greater potency.

In some embodiments, an IdeS variant of the disclosure has human IgG cleaving potency, as determined using ELISA and expressed as an IC50 value that is at least or about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, or 2.5 nM less compared to the IC50 value of wildtype IdeS in the same type of assay, or lower, or an IC50 value between or range encompassing any of the foregoing specifically enumerated values. In some embodiments, an IdeS variant of the disclosure has human IgG cleaving potency, as determined using ELISA and expressed as an IC50 value, of at most 5.5, 5.4, 5.3, 5.2, 5.1, 5.0, 4.9, 4.8, 4.7, 4.6, 4.5, 4.4, 4.3, 4.2, 4.1, 4.0, 3.9, 3.8, or 3.7 nM, or lower, or an IC50 value between or range encompassing any of the foregoing specifically enumerated values.

### Methods of Producing IdeS Proteins

Variant IdeS proteins of the disclosure can be produced using any technique known in the art for producing a recombinant protein. In some embodiments, the coding sequence for an IdeS protein variant can be cloned into an expression vector, which is introduced into a suitable host cell after which the sequence is transcribed and translated into protein. Coding sequences can be optimized for the type of host cell in which they are intended to be expressed. IdeS variant proteins expressed by the host cells can then be purified using techniques known in the art for purifying recombinant proteins.

In some embodiments, the expression vector is a bacterial expression vector comprising a promoter (such as a lac, PL, or T7 promoter) and terminator (such as a T7 terminator) for respectively initiating and terminating transcription of the protein coding sequence, as well as other functional elements, such as multiple cloning site into which the coding sequence can conveniently inserted, a ribosome binding site to increase translation efficiency, a bacterial origin of replication, sequence encoding an affinity tag and protease cleavage site to enable tag removal, and an antibiotic resistance gene (such as for ampicillin or kanamycin). Expression vectors can be replicated in bacterial hosts (such as the DH10B and DH5-alpha strains), purified, and then used to transform other bacterial strains suitable for recombinant protein expression (such as E. coli BL21, BL21 (DE3), and K-12 strains). After transformation with the expression vector, bacteria are grown in media, usually with antibiotic added to prevent growth of untransformed cells. Depending on the nature of the promoter, protein expression can then be induced, such as by adding a nutrient or drug (such as lactose or IPTG where the lac promoter is used) to the media, or changing an environmental variable, such as temperature or pH. Bacteria are then maintained in media under conditions conducive to protein expression. After sufficient time, protein expressed from the vector is harvested and purified. Proteins with a secretion signal may harvested directly from the media. Other proteins, which remain inside bacterial cells, may be harvested after concentrating and lysing the cells, for example using enzymes to digest cell walls, or physical methods, such as sonication. In either case, expressed protein in media or released from cells can be purified using methods familiar to those of ordinary skill in the art, such as filtration to remove cell debris, salt precipitation, chromatographic methods, such as affinity, ion exchange chromatography, or hydrophobic interaction chromatography, and buffer exchange, other methods being possible. Bacterial expression of recombinant proteins is described further, for example, in Langlais, C., Korn, B. (2005). Recombinant Protein Expression in Bacteria. In: Encyclopedic Reference of Genomics and Proteomics in Molecular Medicine. Springer, Berlin, Heidelberg. https://doi.org/10.1007/3-540-29623-9_4800; Overton TW. Recombinant protein production in bacterial hosts. Drug Discov Today. 2014 May;19 (5): 590-601. doi: 10.1016/j.drudis.2013.11.008; Rosano GL and Ceccarelli EA (2014) Recombinant protein expression in Escherichia coli: advances and challenges. Front. Microbiol. 5:172. doi: 10.3389/fmicb.2014.00172; Rosano, G. L., Morales, E. S. and Ceccarelli, E. A. (2019), New tools for recombinant protein production in Escherichia coli: A 5-year update. Protein Science, 28:1412-1422. https://doi.org/10.1002/pro.3668.

In other embodiments, IdeS protein variants of the disclosure may be expressed in non-bacterial host cells, such as yeast cells, plant cells, insect, or mammalian cells, using expression vectors suitable for protein expression in such host cells, and methods familiar to those of ordinary skill in the art.

In addition to IdeS variant proteins, the disclosure provides nucleic acids (e.g., DNA or RNA) comprising nucleotide sequences encoding IdeS protein variants (which may be codon optimized according to the type of host cell in which expression is to be carried out), expression vectors comprising such nucleotide sequences, host cells comprising such expression vectors, methods for producing an IdeS variant protein of the disclosure, as well as methods of purifying such proteins once expressed.

### Methods of Treatment and Prevention

In some embodiments, the disclosure provides methods of administering to a subject in need of prevention or treatment of a disease or disorder characterized by an excessive concentration in the blood, plasma or serum of such subject of certain IgG antibodies, an amount of an IdeS variant protein of the disclosure sufficient to prevent or treat the disease or disorder. In some embodiments, the disease or disorder is treated or prevented by reducing the concentration of IgG antibodies in the blood, plasma or serum of the subject. In some embodiments, the subject is a human subject and the IgG antibodies may be of the IgG1, IgG2, IgG3, or IgG4 subclasses.

As used herein, in reference to a disease or disorder which has manifested in a subject, “treat” or “treatment” means to decrease or arrest its severity or rate of progression, to at least partially alleviate at least one symptom or sign associated with such disease or disorder, or to change the value of a diagnostic assessment or biomarker toward a value which is indicative of a less severe disease state. A therapeutically effective amount of an IdeS variant of the disclosure is one that is sufficient to treat a disease or disorder of a subject.

As used herein, in reference to a disease or disorder which has not yet manifested in a subject, but to which such subject is believed to be susceptible, “prevent” or “prevention” means to prevent or delay (even if temporarily) initiation of a disease process, or initiation of symptoms or signs associated with such disease or disorder. A prophylactically effective amount of an IdeS variant of the disclosure is one that is sufficient to prevent a disease or disorder of a subject.

In some embodiments, the disease or disorder is an autoimmune disorder in which a subject produces IgG antibodies that bind to a self-antigen (autoantibodies), even if unknown, expressed by cells or tissues of the body. Exemplary autoimmune disorders that may be treated using the IdeS variant proteins of the disclosure includes, without limitation, Addison's disease, dermatomyositis, Hashimoto thyroiditis, anti-glomerular basement membrane disease, anti-neutrophil cytoplasmic antibody vasculitis, vasculitis, Wegener granulomatosis, Churg-Strauss syndrome, microscopic polyangiitis, anti-N-methyl-D-aspartate receptor encephalitis, anti-phospholipid antibody syndrome, autoimmune bullous skin disease, pemphigus, pemphigus foliaceus, fogo selvagem, pemphigus vulgaris, autoimmune hemolytic anemia, pernicious anemia, autoimmune hepatitis, autoimmune neutropenia, bullous pemphigoid, celiac disease, chronic utricaria, complete congenital heart block, diabetes type 1, epidermolysis bullosa acquisita, essential mixed cryoglobulinemia, Goodpasture's syndrome, anti-glomerular basement membrane disease, Graves' disease, Basedow's disease, Guillain-Barre syndrome, acute inflammatory demyelinating polyneuropathy, acute motor axonal neuropathy, acquired FVIII deficiency hemophilia, idiopathic thrombocytopenia purpura, Lambert-Eaton myasthenic syndrome, mixed connective tissue disease, myasthenia gravis, dilated cardiomyopathy myocarditis, neuromyelitis optica, primary biliary cirrhosis, multiple sclerosis, systemic sclerosis, CREST syndrome, rheumatic heart disease, rheumatoid arthritis, reactive arthritis, serum-sickness, immune complex hypersensitivity type III, Sjögren syndrome, systemic lupus erythematosus, lupus nephritis, stiff-person syndrome, vitiligo, scleroderma, transplant rejection, and thrombotic thrombocytopenia purpura, others being possible.

In some embodiments, the antigen against which IgG autoantibodies is produced includes, without limitation, Ro-RNP complex, La antigen, small nuclear ribonucleoproteins (snRNP), double stranded DNA, histones, topoisomerase I, centromere, myeloperoxidase, proteinase 3, cardiolipin, citrullinated proteins, carbamylated proteins, rheumatoid factor, phospholipids, alpha 3 chain of basement membrane collagen (type IV collagen), Rh blood group antigens, I antigen, platelet integrin GpIIB: IIIa, epidermal cadherin, ribosomes, pancreatic beta cell antigen, myelin basic protein, carboxypeptidase H, chromogranin A, glutamate decarboxylase, imogen-38, insulin, insulinoma antigen-2 and 2 beta, islet-specific glucose-6-phosphatase catalytic subunit related protein (IGRP), proinsulin, phospholipid-B-2 glycoprotein I complex, poly (ADP-ribose) polymerase, Sm antigens of U-1 small ribonucleoprotein complex, alpha enolase, aquaporin-4, beta arrestin, myelin oligodendrocytic glycoprotein, proteolipid protein, S100 beta, collagen II, heat shock proteins, human cartilage glycoprotein 39, others being possible.

In some embodiments, the disease or disorder is monoclonal gammopathy of undetermined significance (MGUS), in which the paraprotein produced by the abnormal clonal population of plasma cells is IgG.

In some embodiments, a subject with an autoimmune disease and/or that produces autoantibodies, or has MGUS, is administered a dose of an IdeS variant of the disclosure of at least or about 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.35, 0.40, 0.45, or 0.50 mg/kg subject body weight, or a dose or range between and encompassing any of the foregoing specifically enumerated values. In some embodiments, treatment of a subject with an autoimmune disease or MGUS with an IdeS protein variant of the disclosure at the recited dosages is effective to treat or prevent the autoimmune disease or MGUS. In some embodiments, treatment of a subject with an autoimmune disease or MGUS with an IdeS protein variant of the disclosure at the recited dosages is effective to reduce the concentration of total IgG in the subject's blood, plasma or serum by at least or about 60%, 65%, 70%, 75%, 80%, 85%, 90%, 91%, 92%, 93%, 94%, 95%, 96%, 97%, 98%, 99%, or 100%, or a percentage reduction or range between and encompassing any of the foregoing specifically enumerated values, or reduce the concentration of total IgG in the subject's blood, plasma or serum to not more than about 7.0, 6.5, 6.0, 5.5, 5.0, 4.5, 4.0, 3.5, 3.0, 2.5, 2.4, 2.3, 2.2, 2.1, 2.0, 1.9, 1.8, 1.7, 1.6, 1.5, 1.4, 1.3, 1.2, 1.1, 1.0, 0.9, 0.8, 0.7, 0.6, 0.5, 0.4, 0.3, 0.2, 0.1, 0.05 grams per liter (g/L), or less, or a concentration or range between and encompassing any of the foregoing specifically enumerated values. In some embodiments, treatment of a subject with an autoimmune disease with an IdeS protein variant of the disclosure at the recited dosages is effective to inhibit or reduce an effector function of IgG autoantibodies that bind to self-antigen, even if unknown. In some embodiments, the effector function is mediated by an Fc receptor.

In some embodiments, a subject with an autoimmune disease and/or that produces autoantibodies, or has MGUS, may be administered a second or subsequent dose of an IdeS variant of the disclosure, which further dose or doses may be the same or a different dose as the first dose of IdeS variant. In some embodiments, the second or subsequent dose may be administered at any suitable interval so as to maintain an acceptably low level of autoantibodies. The interval may be regular, such as every week, or biweekly, or monthly, or bimonthly, or every 3, 4, 5, or 6 months or some other regular interval. Alternatively, in some embodiments, a subject may be monitored by periodically taking samples, such as of blood, plasma or serum, to determine the concentration or titer of autoantibodies over time, and administering further doses of an IdeS protein variant as desirable or necessary to maintain the concentration or titer of autoantibodies within a predetermined range. In some embodiments, the subject is a human subject and the IgG antibodies may be of the IgG1, IgG2, IgG3, or IgG4 subclasses.

In some embodiments, the disclosure provides methods of administering to a human subject sensitized for human leukocyte antigen (HLA) in need of tissue or organ transplantation an amount of an IdeS variant protein of the disclosure sufficient to reduce or deplete the concentration of anti-HLA IgG antibodies in the blood of such subject, thereby preventing antibody-mediated rejection after transplantation. In some embodiments, the transplanted organ is kidney, liver, heart, pancreas, lung, or intestine, or other organ. In some embodiments, the anti-HLA IgG antibodies may be of the IgG1, IgG2, IgG3, or IgG4 subclasses.

As is known in the art, HLA sensitization can occur when a human has been exposed to another individual's cells, for example as a result of a blood transfusion, pregnancy, or previous organ transplant. Upon such exposure, the immune system of the sensitized individual recognizes the exogenous cells as foreign and produces anti-HLA antibodies. When the titer of such circulating antibodies is sufficiently high, they can cause the sensitized host to reject transplanted tissues or organs through mechanisms of antibody-mediated rejection (AMR), such as antibody dependent cellular cytotoxicity (ADCC) and complement dependent cytotoxicity (CDC).

Whether a subject is sensitized may be determined by any suitable method. For example, a panel reactive antibody (PRA) test may be used to determine if a subject is sensitized. A calculated PRA score at or above a certain threshold means that a subject is “high immunologic risk” or “sensitized,” and is at risk for AMR should transplantation proceed. Alternatively, a cross match test may be conducted in which a sample of the transplant donor's blood is mixed with that of the subject. A positive cross match means that the subject has antibodies that react to the donor sample, indicating that the subject is sensitized and transplantation should not proceed. In some embodiments, a sensitized subject at risk for AMR is that exhibits a calculated panel reactive antibody test score of at least or about 10%, 15%, 20%, 25%, 30%, 35%, 40%, 45%, 50%, 55%, or 60%, or more.

In some embodiments, a sensitized subject is administered a dose of an IdeS variant of the disclosure of at least or about 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.35, 0.40, 0.45, or 0.50 mg/kg subject body weight, or a dose or range between and encompassing any of the foregoing specifically enumerated values, after which said subject undergoes tissue or organ transplant. In some embodiments, treatment of a sensitized subject with an Ides protein variant of the disclosure at the recited dosages is effective to reduce or eliminate the risk of AMR upon tissue or organ transplantation.

In some embodiments, the period between administering the IdeS variant and transplantation is at least or about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 hours, or more, or a period of time or range between and encompassing any of the foregoing specifically enumerated values.

In some embodiments, a sensitized subject may be administered a second or subsequent dose of an IdeS variant of the disclosure prior to transplantation, which further dose or doses may be the same or a different dose as the first dose of IdeS variant. In some embodiments, the second dose is administered at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 hours or more after the first dose, or a period of time or range between and encompassing any of the foregoing specifically enumerated values. In some embodiments, administering more than one dose of an IdeS protein variant may be desirable when a subject produces nAbs against IdeS due to prior infection with S. pyogenes, prior administration of IdeS, presence of particularly high titers of anti-HLA antibodies, as well as other reasons. In some embodiments, prior to a second or subsequent administration of an IdeS protein variant of the disclosure, a subject may be monitored by taking at least one sample, such as of blood, plasma or serum, and testing such sample for the concentration or titer of nAbs or other type of IgG to determine whether further administration of the IdeS protein variant is desirable or necessary.

In some embodiments, the dose of IdeS variant and/or the period between administering the IdeS variant and transplantation is time sufficient for the subject to convert from crossmatch positive to crossmatch negative using an assay to detect presence of donor-specific anti-HLA antibodies (DSAs) in blood, plasma or serum samples from the subject. DSAs in such samples can be quantified with a variety of methods known in the art, such as complement-dependent cytotoxicity (CDC), flow cytometry crossmatch (FCXM), and/or multiplex single antigen bead (SAB) assays. Such methods are described further in, for example, Mulley, W R, and J Kanellis, Understanding crossmatch testing in organ transplantation: A case-based guide for the general nephrologist, Nephrology 16:125-133 (2011) (doi: 10.1111/j.1440-1797.2010.01414.x); Lorant T, et al., Safety, immunogenicity, pharmacokinetics, and efficacy of degradation of anti-HLA antibodies by IdeS (imlifidase) in chronic kidney disease patients, Am J Transplant. 18:2752-2762 (2018) (DOI: 10.1111/ajt.14733).

In some embodiments, the dose of IdeS variant and/or the period between administering the IdeS variant and transplantation is time sufficient for the concentration of total IgG in the subject's blood, plasma or serum to be reduced by at least or about 60%, 65%, 70%, 75%, 80%, 85%, 90%, 91%, 92%, 93%, 94%, 95%, 96%, 97%, 98%, 99%, or 100%, or a percentage reduction or range between and encompassing any of the foregoing specifically enumerated values. In some embodiments, the dose of IdeS variant and/or the period between administering the IdeS variant and transplantation is time sufficient for the concentration of total IgG in the subject's blood, plasma or serum to be reduced to not more than about 7, 6.5, 6, 5.5, 5, 4.5, 4, 3.5, 3, 2.5, 2.4, 2.3, 2.2, 2.1, 2.0, 1.9, 1.8, 1.7, 1.6, 1.5, 1.4, 1.3, 1.2, 1.1, 1.0, 0.9, 0.8, 0.7, 0.6, 0.5, 0.4, 0.3, 0.2, 0.1, 0.05 grams per liter (g/L), or less, or a concentration or range between and encompassing any of the foregoing specifically enumerated values.

In some embodiments, the dose of IdeS variant and/or the period between administering the IdeS variant and transplantation is time sufficient for the concentration of DSAs in the subject's blood, plasma or serum to be reduced by at least or about 60%, 65%, 70%, 75%, 80%, 85%, 90%, 91%, 92%, 93%, 94%, 95%, 96%, 97%, 98%, 99%, or 100%, or a percentage reduction or range between and encompassing any of the foregoing specifically enumerated values. Concentration of DSAs in blood, plasma or serum samples of subjects can be determined using CDC, FCXM, SAB, or any other suitable assay known in the art.

In some embodiments, the dose of IdeS variant and/or the period between administering the IdeS variant and transplantation is time sufficient for the calculated panel reactive antibody (PRA) assay score of the subject's serum to be reduced by at least or about 50%, 55%, 60%, 65%, 70%, 75%, 80%, 85%, 90%, or 95%, or more, or a percentage reduction or range between and encompassing any of the foregoing specifically enumerated values. In some embodiments, the dose of IdeS variant and/or the period between administering the IdeS variant and transplantation is time sufficient for the calculated panel reactive antibody (PRA) assay score of the subject's serum to be reduced to not more than about 50%, 45%, 40%, 35%, 30%, 25%, 20%, 19%, 18%, 17%, 16%, 15%, 14%, 13%, 12%, 11%, 10%, 9%, 8%, 7%, 6%, 5%, 4%, 3%, 2%, or 1%, or less, or concentration or range between and encompassing any of the foregoing specifically enumerated values. The calculated panel reactive antibody (PRA) assay score of a subject's serum can be determined using a complement-dependent cytotoxicity (CDC) assay, or any other suitable assay known in the art.

In some embodiments, a sensitized subject that received a transplant may be administered a second or subsequent dose of an IdeS protein variant of the disclosure after transplantation, which further dose or doses may be the same or a different dose as the first dose of IdeS variant administered prior to transplantation. In some embodiments, the second dose is administered at least 12, 24, 36, or 48 hours, or 5, 6, 7, 8, 9, 10, 11, 12, or 13 days, or 2, 3, or 4 weeks, or more, after transplantation, or a period of time or range between and encompassing any of the foregoing specifically enumerated values. In some embodiments, prior to a second or subsequent administration of an IdeS protein variant of the disclosure, a subject may be monitored by taking at least one sample, such as of blood, plasma or serum, and testing such sample for the concentration or titer of nAbs or other type of IgG to determine whether further administration of the IdeS protein variant is desirable or necessary.

In some embodiments, IdeS variants of the disclosure may be used to reduce the concentration of neutralizing IgG antibodies in subjects prior to gene therapy. In certain methods of gene therapy, naturally occurring viruses are engineered by modifying their genomes both to prevent replication as well as introduce heterologous genetic information sufficient to express RNA or protein with desired function in target cells of a subject. Such modified viruses are called vectors in recognition of their function to carry the novel genetic information into target cells of patients for the purpose of effecting therapy. Examples of types of viruses which have been used to engineer gene therapy vectors include adenovirus, adeno-associated virus, and lentivirus (such as HIV-1 or HIV-2).

As is known in the art, recombinant AAV vectors are derived from naturally occurring adeno-associated viruses, but are heavily modified both to prevent their replication as well as to include heterologous non-viral gene sequences intended to confer a therapeutic effect. AAV vectors typically comprise an AAV capsid and a modified AAV genome. The capsid is the proteinaceous shell comprising three viral proteins, AAV VP1, VP2, and VP3, responsible for specific binding to target cells, as well as enclosing and protecting the genome. AAV vectors may employ capsids from any naturally occurring serotype or variant, as well as non-naturally occurring capsids which have been derivatized, modified, engineered, or evolved to improve their function in some way relative to naturally occurring parental capsid or capsids. In turn, the vector genome is derived from the AAV viral genome, but has been heavily modified. More specifically, the only viral sequences retained are the inverted terminal repeats (ITRs), one of which is positioned at each of the 5′ and 3′ termini of the single stranded DNA molecule which represents the genome. Entirely removed, however, are the AAV viral rep and cap genes, respectively responsible for encoding four Rep proteins (in AAV2), which are involved in such critical functions as replication and packaging, and the three viral capsid proteins. In vectors, these genes are replaced by a transgene expression cassette. The latter sequence is intended to express in transduced target cells a transgene product intended to have some therapeutic effect. In some vectors, the transgene encodes for a protein that is missing or defective in a patient due to an underlying genetic mutation, and treatment is intended to provide a functional copy of the gene. For example, the transgene can encode for clotting factor VIII, which is missing or defective in hemophilia A, or for clotting factor IX, which is missing or defective in hemophilia B. Numerous other types of protein encoding transgenes useful in AAV vectors are possible. In other vectors, the transgene can encode an RNA or protein intended to alter the function of a transduced target cell. Examples include regulatory RNAs, such as those involved in the mechanism of RNA interference, or a Cas nuclease expressed for purposes of effecting gene editing. To support expression of the transgene, expression cassettes also often include transcription control sequences active in the target cell, such as a promoter to initiate transcription of the transgene and a polyadenylation signal sequence to terminate transcription. As known in the art, expression cassettes may include other functional subsequences, such as enhancers, introns, stuffers, miRNA sequences, intended to improve desired properties, such as expression levels, tissue specificity of expression, message stability, packaging fidelity, and others. AAV vectors may include other features intended to improve or affect function in some desired way, such as use of self complementary genomes. The type of AAV vector used in connection with the methods of treatment or prevention described herein should not be considered limiting.

Further information about AAV vectors can be found, for example, in Pupo, A, et al., AAV vectors: The Rubik's cube of human gene therapy, Mol Ther 30 (12): 3515-41 (2022) (doi.org/10.1016/j.ymthe.2022.09.015); Li, C and RJ Samulski, Engineering adeno-associated virus vectors for gene therapy, Nat Revs Genetics 21:255-72 (2020) (doi.org/10.1038/s41576-019-0205-4); Wang, D, et al., Adeno-associated virus vector as a platform for gene therapy delivery. Nat Rev Drug Discov 18, 358-378 (2019) (doi.org/10.1038/s41573-019-0012-9); Bulcha, J T, et al., Viral vector platforms within the gene therapy landscape. Sig Transduct Target Ther 6, 53 (2021) (doi.org/10.1038/s41392-021-00487-6). Further information about lentiviral vectors can be found, for example, in Wolff, J H and Mikkelsen, JG, Delivering genes with human immunodeficiency virus-derived vehicles: still state-of-the-art after 25 years. J Biomed Sci 29, 79 (2022) (doi.org/10.1186/s12929-022-00865-4); Poletti, V and Mavilio, F. Designing Lentiviral Vectors for Gene Therapy of Genetic Diseases. Viruses. 2021 Aug. 2;13 (8): 1526 (doi: 10.3390/v13081526).

One challenge to using modified viruses as gene therapy vectors is that candidates for gene therapy may have been earlier infected with the same or similar type of virus from which the vector is fashioned, and generated an antibody response against one or more components of the virus which are retained in the vector. Depending on the titer and specificity of the antibodies, their presence in a gene therapy candidate can interfere with the ability of vector particles to bind target cells, preventing target cell transduction and expression of the therapeutic transgene carried by the vectors. At best, this can reduce the effective dose of the vector, requiring higher doses to compensate, or at worst render the intended gene therapy ineffective so that the candidate must be excluded from treatment. For example, numerous adeno-associated virus (AAV) serotypes and variants naturally infect humans causing some to seroconvert and produce neutralizing antibodies (nAbs) against capsid proteins used in certain AAV-base vectors. This phenomenon is described, for example, in Rasko, J, et al., Global Seroprevalence of Neutralizing Antibodies Against Adeno-Associated Virus (AAV) Serotypes of Relevance to Gene Therapy, Blood (2022) 140 (Supplement 1): 10668-10670 (doi.org/10.1182/blood-2022-158305); Kruzik, A, et al., Prevalence of Anti-Adeno-Associated Virus Immune Responses in International Cohorts of Healthy Donors, Mol Ther Meths Clin Devel, Vol. 14, P126-133 (2019) (doi.org/10.1016/j.omtm.2019.05.014); Weber, T, Anti-AAV Antibodies in AAV Gene Therapy: Current Challenges and Possible Solutions, Front Immunol, 12:658399 (2021) (doi: 10.3389/fimmu.2021.658399).

Various strategies have been proposed for mitigating the challenge of nAbs against AAV vectors, such as treating gene therapy candidates with drugs to suppress B cell function, but one approach with particular promise involves administering IgG-degrading enzymes such as IdeS or IdeZ to patients before administering gene therapy vector. Such approaches have been described, for example, in Leborgne C, et al., IgG-cleaving endopeptidase enables in vivo gene therapy in the presence of anti-AAV neutralizing antibodies. Nat Med. (2020) 26:1096-101. doi: 10.1038/s41591-020-0911-7; Elmore Z C, et al., Rescuing AAV gene transfer from neutralizing antibodies with an IgG-degrading enzyme. JCI Insight. (2020) 5: e139881. doi: 10.1172/jci.insight.139881. Due to their superior properties compared to the wildtype IdeS used in earlier experiments, IdeS variants of the disclosure may advantageously be employed to reduce the concentration of neutralizing IgG antibodies in the blood of candidates for gene therapy sufficiently so that those who might otherwise be ineligble can be successfully treated with vector.

Accordingly, in some embodiments, the disclosure provides a method of preparing or pretreating a subject for gene therapy, wherein the subject has a pre-existing titer of neutralizing IgG antibodies against a component of a gene therapy vector sufficiently high as to interfere with, reduce or block the ability of the vector to transduce its intended target cells if administered to such subject, by administering to such subject an amount of an Ides protein variant of the disclosure sufficient to reduce or eliminate, at least temporarily, the concentration of nAbs to a level that would no longer interfere with, reduce or block vector transduction in the subject. In some embodiments, such treatment is effective to enable successful administration of a gene therapy vector to a subject that would otherwise be deemed ineligible for gene therapy due to the presence of an excessively high titer of nAbs against a component of the gene therapy vector. In some embodiments, such treatment is effective to reduce the concentration of nAbs in the subject's blood as to enable a gene therapy vector to successfully transduce intended target cells after its subsequent administration. In some embodiments, the subject is gene therapy treatment naïve, meaning that the subject had never before received a particular type of gene therapy or been administered a particular type of gene therapy vector.

The titer of pre-existing neutralizing antibodies in serum samples from a subject can be determined using a variety of assays known in the art. Generally, such assays are designed to detect what dilution of a serum or other sample believed to contain nAbs is effective to inhibit 50% of some physiologically relevant signal produced by the assay in the absence of any added sample, which is defined as 100%. For example, assays have been designed to detect and quantify nAbs against particular capsids of recombinant AAV vectors where the signal is amount of a reporter protein (such as luciferase) produced by transduced cells in vitro. Serial dilutions of the serum sample are mixed with a predetermined amount of the reporter vector using the capsid in question. After incubation, the mixtures are added to cells in culture known to support vector binding and expression of the reporter transgene. As expression proceeds, the amount of signal from the reporter (for example, light output from luciferase) is indicative of transduction efficiency, which is inversely proportional to the amount of nAb in the mixture. From a graph relating sample dilution factor to signal intensity, the dilution factor resulting in 50% signal inhibition compared to no added serum is defined as the nAb titer. Similar assays have been designed which quantify the number of vector particles (as genome copies) binding to target cells in culture after exposure to diluted serum samples. The dilution factor preventing 50% of vector particles from binding to target cells is then defined as the titer. Assays are described further, for example, in Meliani, A, et al., Determination of anti-adeno-associated virus vector neutralizing antibody titer with an in vitro reporter system. Hum. Gene Ther. Methods 26, 45-53 (2015) (doi.org/10.1089/hgtb.2015.037); Guo, P, et al., Rapid AAV-Neutralizing Antibody Determination with a Cell-Binding Assay, Mol Ther Meths Clin Devel. 13:40-46 (2019) (doi.org/10.1016/j.omtm.2018.11.007).

In some embodiments, a subject to be pretreated with an IdeS protein variant of the disclosure prior to administration of a gene therapy vector has a neutralizing antibody titer to the vector of at least or about 1:2, 1:3, 1:4, 1:5, 1:6, 1:7, 1:8, 1:9, 1:10, 1:15, 1:20, 1:25, 1:30, 1:40, 1:50, 1:60, 1:70, 1:80, 1:90, 1:100, 1:120, 1:125, 1:150, 1:175, 1:200, 1:250, 1:300, 1:350, 1:400, 1:450, 1:500, 1:550, 1:600, 1:650, 1:700, 1:750, 1:800, 1:850, 1:900, 1:950, 1:1000, 1:1100, 1:1200, 1:1300, 1:1400, 1:1500, 1:1600, 1:1700, 1:1800, 1:1900, 1:2000, 1:2100, 1:2200, 1:2300, 1:2400, 1:2500, 1:2600, 1:2700, 1:2800, 1:2900, or 1:3000, or higher titer, or a titer or range between and encompassing any of the foregoing specifically enumerated values. In the foregoing, the ratio indicates the serum sample dilution factor calculated to inhibit 50% of gene therapy vector activity in a suitable assay, such as a transduction efficiency assay employing a reporter vector, or a vector and target cell binding assay, or other suitable assay. Furthermore, in the foregoing, greater dilutions, as indicated by larger values for the second figure in the ratio, indicates higher concentration of nAbs in the sample, and therefore higher titer of such nAbs.

In some embodiments, the nAbs are specific for a component of a recombinant viral vector, such as an adenoviral vector, an adeno-associated viral vector, or a lentiviral vector, or another vector derived from a virus. In some embodiments, the nAbs are specific for a protein comprising the capsid of an AAV vector, such as an AAV VP1, VP2, or VP3 capsid protein. In some embodiments, the capsid recognized by the nAbs can be from any AAV naturally occurring serotype or variant, non-limiting examples of which include AAV1, AAV2, AAV3B, AAV4, AAV5, AAV6, AAV7, AAV8, AAV9, AAV10, AAV11, AAV12, AAV13, AAVRh74, or AAVRh10, or others, or from any non-naturally occurring modified, engineered, or evolved AAV, or AAV derivative, non-limiting examples of which include those designated AAV-2i8, Spark 100, AAV-Voy801, AAV-DJ, AAV PHP.B, or numerous others.

In some embodiments, a subject with pre-existing titer of neutralizing IgG antibodies against a component of a gene therapy vector sufficiently high as to interfere with, reduce or block the ability of the vector to transduce its intended target cells is prepared or pretreated for gene therapy with the vector by administering to such subject a dose of an IdeS variant of the disclosure effective to reduce the concentration of nAbs to a level that no longer interferes with, reduces or blocks vector transduction in the subject. In some embodiments, the effective dose of an IdeS protein variant of the disclosure is at least or about 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.35, 0.40, 0.45, or 0.50 mg/kg subject body weight, or a dose or range between and encompassing any of the foregoing specifically enumerated values. In some embodiments, treating a subject previously administered a gene therapy vectory with an IdeS protein variant of the disclosure at the recited dosages is effective to inhibit or reduce an effector function of neutralizing IgG antibodies produced by the subject against a component of a gene therapy vector.

In some embodiments, pretreating a subject with a pre-existing nAb titer against a gene therapy vector is effective to reduce the titer of nAbs against the vector in the subject's blood, plasma or serum by at least or about 60%, 65%, 70%, 75%, 80%, 85%, 90%, 91%, 92%, 93%, 94%, 95%, 96%, 97%, 98%, 99%, or 100%, or a percentage reduction or range between and encompassing any of the foregoing specifically enumerated values. In some embodiments, pretreating a subject with a pre-existing nAb titer against a gene therapy vector is effective to reduce the titer of nAbs against the vector in the subject's blood, plasma or serum to a value of not more than 1:10, 1:9, 1:8, 1:7, 1:6, 1:5, 1:4, 1:3, 1:2, 1:1, 1:0.9, 1:0.8, 1:0.7, 1:0.6, 1:0.5, 1:0.4, 1:0.3, 1:0.2, 1:0.1, or less, or a titer or range between and encompassing any of the foregoing specifically enumerated values.

In some embodiments, the methods of preparing or pretreating a subject with a pre-existing nAb titer against a gene therapy vector comprise administering a single dose of an IdeS protein variant of the disclosure to such subject. In other embodiments, the methods comprise administering a plurality of doses of an IdeS variant protein, such as 2, 3, 4 or more doses of such IdeS variant protein. Each dose of the plurality of doses can be the same dose or a different dose. The time period between each of the several doses, of more than two, can be the same time period or a different time period. Exemplarily non-limiting periods between administering any two doses of IdeS protein variant includes 1, 2, 3, 4, 5, 6, 7, 8, 9, 10, 11, 12, 13, 14, 15, 16, 17, 18, 19, 20, 22, 24, 25, 26, 28, 30, 32, 34, 35, 36, 38, 40, 42, 44, 45, 46, 48 hours, or more, or a period of time or range between and encompassing any of the foregoing specifically enumerated values. In some embodiments, administering more than one dose of an IdeS protein variant may be desirable when a subject produces nAbs against IdeS due to prior infection with S. pyogenes, prior administration of IdeS, presence of particularly high titers of antibodies against the gene therapy vector, as well as other reasons. In some embodiments, prior to a second or subsequent administration of an IdeS protein variant of the disclosure, a subject will be monitored by taking at least one sample, such as of blood, plasma or serum, and testing such sample for the concentration or titer of nAbs or other type of IgG to determine whether further administration of the IdeS protein variant is desirable or necessary.

In some embodiments, the methods of the disclosure further comprise, after pretreating a subject with a sufficient dose an an IdeS protein variant of the disclosure so as to reduce nAb titer against a gene therapy vector, a second step of administering such gene therapy vector to such subject. In some embodiments, the period between administering the IdeS variant (or last dose thereof if more than one) and administering the gene therapy vector is at least or about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 50, 52, 54, 55, 56, 58, 60, 62, 64, 65, 66, 68, 70, 72, 74, 75, 76, 78, 80, 84, 85, 90, 95, 96, 100, 108, 110, 120, 125, 130, 132, 135, 140, 144, 145, 150, 155, 156, 160, 165, 168 hours, or 8, 9, 10, 11, 12, 13, 14 days, or more time, or a period of time or range between and encompassing any of the foregoing specifically enumerated values.

In some embodiments, in the second step of administering a gene therapy vector to a subject, the gene therapy vector can be any suitable gene therapy vector given the nature and severity of the subject's underlying health condition, such as diagnosis with a disease or disorder amenable to treatment or prevention using a gene therapy vector. Choice of which gene therapy vector is to be administered is within the knowledge of those ordinarily skilled, as will other aspects of successfully administering such treatment with the goal of achieving a desired therapeutic or prophylactic outcome, such as subject selection and application of any exclusion criteria for treatment, the dose and dose form of the gene therapy vector, the number and frequency of dose administrations, the route of administration, management of adverse events or side effects (such as immune reaction against the vector), and follow-up, such as monitoring the subject for changes in health status indicative of efficacy or side effects, assessment of clinical endpoints, or taking tissue or fluid samples to detect and measure changes in biomarkers, or other steps. In some embodiments, the gene therapy vector is a recombinant AAV vector comprising any suitable capsid and transgene expression cassette given the nature of the subject's underlying health condition, disease or disorder, and other considerations familiar to those of ordinary skill in the art. In some embodiments, administering an IdeS protein variant of the disclosure before gene therapy is effective to reduce the dose of the gene therapy vector that would otherwise be required to be administered to a subject to achieve a given level of transduction efficiency, level of therapeutic or prophylactic efficacy, or to express a given level of transgene product.

In some embodiments, an AAV vector expresses a functional clotting factor VIII protein for treatment of hemophilia A, or a functional clotting factor IX protein for treatment of hemophilia B, or a miniaturized but functional (at least partially) version of the dystrophin protein for treatment of Becker or Duchenne muscular dystrophy. Use of other AAV vectors expressing other types of transgenes to treat or prevent other types of diseases or disorders is also possible. In some other embodiments, the gene therapy vector is a different recombinant viral vector, such as adenovirus (e.g., AdV5), lentivirus (e.g., HIV-1, HIV-2), retrovirus, herpes simplex virus (e.g., HSV1, HSV2), alphavirus, favivirus, others being possible.

In some embodiments, the methods of the disclosure further comprise, after pretreating a subject with an IdeS protein variant of the disclosure so as to reduce nAb titer against a gene therapy vector and then administering a gene therapy vector, a third step of administering to the subject one or more doses of an IdeS protein variant of the disclosure so as to maintain a low level of nAbs in the subject. In some embodiments, a dose of an IdeS protein variant to be administered after administering the gene therapy vector is at least or about 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.35, 0.40, 0.45, or 0.50 mg/kg subject body weight, or a dose or range between and encompassing any of the foregoing specifically enumerated values, which is effective to maintain a low or undetectable level of nAbs in the subject. In some embodiments, the IdeS protein variant is administered to a subject at least or about 3, 6, 9, 12, 15, 18, 24, 30, 36, 42, 48, 54, 60, 66, 72 hours, or 3.5, 4, 4.5, 5, 5.5, 6, 7, 8, 9, 10, 11, 12, 13, 14 days, or 2.5, 3, 3.5, 4 weeks, after administration of the gene therapy vector, or more time, or a period of time or range between and encompassing any of the foregoing specifically enumerated values, which is effective to maintain a low or undetectable level of nAbs in the subject.

In some embodiments, the methods of post-treating a subject after gene therapy to maintain low or undetectable levels of nAbs comprise administering a single dose of an IdeS protein variant of the disclosure to such subject. In other embodiments, the methods comprise administering a plurality of doses of an IdeS variant protein, such as 2, 3, 4 or more doses of such IdeS variant protein. Each dose of the plurality of doses can be the same dose or a different dose. The time period between each of the several doses, of more than two, can be the same time period or a different time period. Exemplary non-limiting periods between administering any two doses of IdeS protein variant includes 1, 2, 3, 4, 5, 6, 7, 8, 9, 10, 11, 12, 13, 14, 15, 16, 17, 18, 19, 20, 22, 24, 25, 26, 28, 30, 32, 34, 35, 36, 38, 40, 42, 44, 45, 46, 48 hours, or more, or a period of time or range between and encompassing any of the foregoing specifically enumerated values. In some embodiments, prior to a second or subsequent administration of an IdeS protein variant of the disclosure, a subject may be monitored by taking at least one sample, such as of blood, plasma or serum, and testing such sample for the concentration or titer of nAbs or other type of IgG to determine whether further administration of the IdeS protein variant is desirable or necessary.

In other embodiments, the methods of the disclosure allow readministering or redosing a gene therapy vector to a subject. On occasion, in some subjects, a first treatment with gene therapy may produce a suboptimal therapeutic effect or therapeutic effect of supoptimal duration. Different causes are possible. For example, the transduction efficiency of target cells may not be as high as desired due to presence of nAbs to a vector component; the cellular immune system gradually eliminates transduced cells; gene therapy was administered to a pediatric subject and as the subject matures organ and body size increases so that the relative amount of the therapeutic transgene product from the vector gradually declines, or for some other reason. In such circumstances, it may be desirable to readminister to the subject the same or similar type of vector at least a second time. One challenge to successful redosing, however, arises from the fact that the first administration of a gene therapy vector can stimulate an immune response against one or more of its components, including the production of nAbs which could prevent a second administration of a gene therapy vector from being successful. Due to their demonstrated efficacy in cleaving and inactivating IgG antibodies, the disclosure also provides, in some embodiments, methods of administering an IdeS protein variant to a subject previously treated with a gene therapy vector against which the subject produced neutralizing IgG antibodies so as to reduce or eliminate the titer of such antibodies, thereby allowing a second or subsequent administration of the same type of vector, or different vector which nevertheless shares antigenic features of the first vector so as to be recognized by at least some of the same nAbs.

In some embodiments, a subject to be treated with an IdeS protein variant of the disclosure before being readministered a gene therapy vector has a neutralizing antibody titer to the vector of at least or about 1:2, 1:3, 1:4, 1:5, 1:6, 1:7, 1:8, 1:9, 1:10, 1:15, 1:20, 1:25, 1:30, 1:40, 1:50, 1:60, 1:70, 1:80, 1:90, 1:100, 1:120, 1:125, 1:150, 1:175, 1:200, 1:250, 1:300, 1:350, 1:400, 1:450, 1:500, 1:550, 1:600, 1:650, 1:700, 1:750, 1:800, 1:850, 1:900, 1:950, 1:1000, 1:1100, 1:1200, 1:1300, 1:1400, 1:1500, 1:1600, 1:1700, 1:1800, 1:1900, 1:2000, 1:2100, 1:2200, 1:2300, 1:2400, 1:2500, 1:2600, 1:2700, 1:2800, 1:2900, 1:3000, or higher titer, or a titer or range between and encompassing any of the foregoing specifically enumerated values.

In some embodiments, the gene therapy vector to be readministered is a recombinant viral vector, such as an adenoviral vector, an adeno-associated viral (AAV) vector, or a lentiviral vector, or another vector derived from a virus. In some embodiments, the gene therapy vector to be readministered is an AAV vector which uses the same type of capsid as the initially administered vector, or a different capsid recognized by the same nAbs produced against the initially administered vector's capsid.

In some embodiments, before being readministered a gene therapy vector against which a subject produced nAbs resulting from prior gene therapy of sufficient titer as to interfere with, reduce or block the ability of the readministered vector to transduce its intended target cells, a subject is administered a dose of an IdeS protein variant of the disclosure effective to reduce the concentration of nAbs to a level that no longer interferes with, reduces or blocks vector transduction in the subject. In some embodiments, an effective dose of an IdeS variant of the disclosure is at least or about 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.35, 0.40, 0.45, or 0.50 mg/kg subject body weight, or a dose or range between and encompassing any of the foregoing specifically enumerated values.

In some embodiments, treating a subject with an IdeS protein variant of the disclosure is effective to reduce the titer of nAbs against the vector to be readministered by at least or about 60%, 65%, 70%, 75%, 80%, 85%, 90%, 91%, 92%, 93%, 94%, 95%, 96%, 97%, 98%, 99%, or 100%, or a percentage reduction or range between and encompassing any of the foregoing specifically enumerated values. In some embodiments, treating a subject with an IdeS protein variant of the disclosure is effective to reduce the titer of nAbs against the vector to be readministered to a value of not more than 1:10, 1:9, 1:8, 1:7, 1:6, 1:5, 1:4, 1:3, 1:2, 1:1, 1:0.9, 1:0.8, 1:0.7, 1:0.6, 1:0.5, 1:0.4, 1:0.3, 1:0.2, 1:0.1, or less, or a titer or range between and encompassing any of the foregoing specifically enumerated values.

In some embodiments, treating a subject with an IdeS protein variant before readministering a gene therapy vector comprises administering a single dose of an Ides protein variant to such subject, whereas in other embodiments, treatment comprises administering a plurality of doses of an IdeS variant protein, such as 2, 3, 4 or more doses of such IdeS variant protein. Each dose of the plurality of doses can be the same dose or a different dose. The time period between each of the several doses, of more than two, can be the same time period or a different time period. Exemplarly non-limiting periods between administering any two doses of IdeS protein variant includes 1, 2, 3, 4, 5, 6, 7, 8, 9, 10, 11, 12, 13, 14, 15, 16, 17, 18, 19, 20, 22, 24, 25, 26, 28, 30, 32, 34, 35, 36, 38, 40, 42, 44, 45, 46, 48 hours, or more, or a period of time or range between and encompassing any of the foregoing specifically enumerated values. In some embodiments, prior to a second or subsequent administration of an IdeS protein variant of the disclosure, a subject may be monitored by taking at least one sample, such as of blood, plasma or serum, and testing such sample for the concentration or titer of nAbs or other type of IgG to determine whether further administration of the IdeS protein variant is desirable or necessary.

In some embodiments, the methods of the disclosure further comprise, after administering to a subject an IdeS protein variant of the disclosure in an amount sufficient to reduce the titer of nAbs against a gene therapy vector with which such subject had initially been administered, a second step of readministering to such subject the same type of gene therapy vector, or administering a distinct vector (such as an AAV vector using a different capsid) which nevertheless is recognized by nAbs produced by the subject against the initially administered gene therapy vector. In some embodiments, the period between administering the IdeS variant (or last dose thereof if more than one) and readministering the gene therapy vector is at least or about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 50, 52, 54, 55, 56, 58, 60, 62, 64, 65, 66, 68, 70, 72, 74, 75, 76, 78, 80, 84, 85, 90, 95, 96, 100, 108, 110, 120, 125, 130, 132, 135, 140, 144, 145, 150, 155, 156, 160, 165, 168 hours, or 8, 9, 10, 11, 12, 13, 14 days, or more time, or a period of time or range between and encompassing any of the foregoing specifically enumerated values.

In some embodiments, the period between the initial administration of gene therapy vector and the readministration of gene therapy vector is at least or about 0.5, 1, 2, 3, 4, 5, 6, 7, 8, 9, 10, or more years, or 1, 2, 3, 4, 5 or more decades, or a period of time or range between and encompassing any of the foregoing specifically enumerated values. In some embodiments, a gene therapy vector is readministered to a subject at least a second time, before which the subject is treated with an IdeS protein variant of the disclosure in an amount effective to reduce the concentration of nAbs against the vector to a level that no longer interferes with, reduces or blocks vector transduction in the subject.

After receiving gene therapy, certain subjects may produce an immune response against the product of the gene therapy vector, such as the product of a therapeutic transgene comprised by the vector. As a result, a subject receiving gene therapy may experience a treatment effect that is suboptimal, or of limited duration. To address these challenges, further provided are methods of administering to subjects that have been treated with a gene therapy vector an IdeS protein variant of the disclosure to treat or prevent an antibody response against the product expressed by the gene therapy vector.

In some embodiments, a subject previously administered a gene therapy vector is administered a dose of an IdeS variant of the disclosure of at least or about 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.35, 0.40, 0.45, or 0.50 mg/kg subject body weight, or a dose or range between and encompassing any of the foregoing specifically enumerated values. In some embodiments, treatment with the IdeS protein variant at the recited dosages is effective to treat or prevent an antibody response against the product expressed by the gene therapy vector. In some embodiments, treating a subject previously administered a gene therapy vectory with an IdeS protein variant of the disclosure at the recited dosages is effective to inhibit or reduce an effector function of neutralizing IgG antibodies produced by the subject against the product expressed by the gene therapy vector.

In some embodiments, treatment of a subject previously adminstered a gene therapy vector with an IdeS protein variant of the disclosure at the recited dosages is effective to reduce the titer of nAbs against the product expressed by the gene therapy vector in the subject's blood, plasma or serum by at least or about 60%, 65%, 70%, 75%, 80%, 85%, 90%, 91%, 92%, 93%, 94%, 95%, 96%, 97%, 98%, 99%, or 100%, or a percentage reduction or range between and encompassing any of the foregoing specifically enumerated values, or reduce the titer of nAbs against the product expressed by the gene therapy vector to a value of not more than 1:10, 1:9, 1:8, 1:7, 1:6, 1:5, 1:4, 1:3, 1:2, 1:1, 1:0.9, 1:0.8, 1:0.7, 1:0.6, 1:0.5, 1:0.4, 1:0.3, 1:0.2, 1:0.1, or less, or a titer or range between and encompassing any of the foregoing specifically enumerated values.

In some embodiments, a subject previously adminstered a gene therapy vector may be administered a second or subsequent dose of an IdeS variant of the disclosure, which further dose or doses may be the same or a different dose as the first dose of IdeS variant. In some embodiments, the second or subsequent dose may be administered at any suitable interval so as to maintain an acceptably low level of nAbs against the product expressed by the gene therapy vector in the subject's blood, plasma or serum. The interval may be regular, such as every week, or biweekly, or monthly, or bimonthly, or every 3, 4, 5, or 6 months or some other regular interval. Alternatively, in some embodiments, a subject may be monitored by periodically taking samples, such as of blood, plasma or serum, to determine the concentration or titer over time of antibodies against the product expressed by the gene therapy vector, and administering further doses of an IdeS protein variant as desirable or necessary to maintain the concentration or titer of nAbs within a predetermined range. In some embodiments, the subject is a human subject and the IgG antibodies may be of the IgG1, IgG2, IgG3, or IgG4 subclasses.

In some embodiments, administration of an IdeS protein variant of the disclosure for purposes of treatment or prophylaxis as described herein may be performed in conjunction with at least a second type of immunosuppresive therapy. In some embodiments, the second type of immunosuppresive therapy can suppress the innate immune system, B cell activity or function, T cell activity or function, or some other attribute or aspect of the immune system, such as complement activity, antigen presentation, or some other aspect of immune function. The second type of immunosuppresive therapy can be non-specific, such as treatment with steroids, or specific, such as treatment with a monoclonal antibody targeting a specific cell surface protein involved in an immune response mechanism, one example being rituximab. In some embodiments, an IdeS protein variant of the disclosure is administered to a subject prior to, substantially contemporaneously with, or after administration to the subject of the second type of immunosuppresive therapy. Often, but not necessarily, the second type of immunosuppresive therapy will be administered first, before administration of an IdeS protein variant, where such first immunosuppresive therapy relies on administering an immunosuppresive agent, such as a monoclonal IgG antibody, that would itself be cleaved in the presence of the IdeS protein variant. Exemplary, non-limiting examples of immunosuppresive agents that may be administered in conjunction with IdeS protein variants of the disclosure include corticosteroids, such as prednisone, calcineurin inhibitors, such as tacrolimus or cyclosporine, IMDH inhibitors, such as azathioprine, leflunomide, or mycophenolate, JAK inhibitors such as tofacitinib, mTOR inhibitors, such as sirolimus, everolimus, or rapamycin, or others, such as abatacept, adalimumab, anakinra, basiliximab, certolizumab, daclizumab, etanercept, golimumab, infliximab, Ixekizumab, natalizumab, rituximab, secukinumab, Tocilizumab, ustekinumab, or vedolizumab, others being possible.

### Subjects

The subject to which an IdeS protein variant of the disclosure may be administered as described herein may be any suitable subject. In some embodiments, such subjects include, without limitation, humans as well as non-human primates, such as gibbons, gorillas, chimpanzees, orangutans, or macaques, and other mammals or animals, such as a domestic animal (e.g., dogs and cats), a farm animal (e.g., poultry such as chickens and ducks, or horses, cows, goats, sheep, pigs), and experimental animals (e.g., mouse, rat, rabbit, guinea pig). Human subjects can be of any age, such as fetal, neonatal, infant, pediatric, juvenile and adult.

### Pharmaceutical Compositions

The disclosure further provides pharmaceutical compositions comprising an Ides protein variant of the disclosure and at least one pharmaceutically acceptable excipient, vehicle, carrier, or diluent. Formulation of such compositions is within the knowledge of those ordinarily skilled, taking into considerations such factors as storage conditions, stability, dose and dosage form, convenience, route of administration, and other factors familiar to those of ordinary skill. Exemplary non-limiting excipients include pH buffers, such as TRIS, phosphate, or citrate; antioxidants or reducing agents, such as ascorbic acid, methionine, or histidine; preservatives, such as benzalkonium chloride; stabilizers, such as PEG, albumin, gelatin, or polyvinylpyrrolidone; monosaccharides, disaccharides, or other carbohydrates, such as glucose, sucrose, trehalose, mannose, or dextrans; chelating agents, such as EDTA; sugar alcohols, such as mannitol or sorbitol; inorganic or organic salts; wetting agents, such as glycerol; and surfactants, such as Tween, Triton, or Pluronic. Exemplary non-limiting diluents include water, isotonic saline, PBS, or Ringer's solution. In some embodiments, pharmaceutical compositions may be provided in lyophilized form for later reconstitution with a diluent, as an aqueous suspension or solution, as liposomal preparations, or as biodegradable polymer systems. Pharmaceutical compositions may be formulated for instantaneous or sustained release. Pharmaceutical compositions of the disclosure may be packaged in any suitable single or multi-use container or enclosure for long term storage, distribution to end users, or convenient administration to subjects, non-limiting examples including vials and plastic IV bags.

### Methods of Administration

The disclosure further provides methods of administering pharmaceutical compositions of the disclosure to a subject by any suitable route of administration, including systemic, regional, and local routes of delivery. Non-limiting examples include administering pharmaceutical compositions by intravenous injection or infusion continuously, or in one or more boluses. Other examples include delivery of pharmaceutical compositions comprising IdeS protein variants to subjects by routes such as parenteral, intradermal, subcutaneous, percutaneous, intramuscular, intra-arterial, intraperitoneal, intra-articular, intraosseous, intrathecal, intraorbital, intramucosal, intraparenchymal, intrapleural, intrahepatic, via the portal vein, or any other suitable route of administration.

### Kits

The disclosure further provides kits, including packaging material and one or more components therein. A kit may include a label or packaging insert including a description of the components or instructions for use in vitro, in vivo, or ex vivo, of the components therein. A kit may contain a collection of such components, e.g., a vial containing a pharmaceutical formulation comprising an IdeS protein variant of the disclosure, a separate vial containing a diluent, and a tube and needle for infusion. In some embodiments, a kit refers to a physical structure housing one or more components of the kit. Packaging material can maintain the components sterilely and can be made of material commonly used for such purposes (e.g., paper, corrugated fiber, glass, plastic, foil, ampules, vials, tubes, etc.). Labels or inserts can variously include information identifying one or more components therein; dose amounts; clinical pharmacology of the active ingredient(s), including mechanism of action, pharmacokinetics, and pharmacodynamics; indications; potential therapeutic or prophylactic benefit of an active ingredient, composition, or component of the kit; potential adverse side effects, complications, or reactions; manufacturer; manufacturing location and date; lot number; expiration date; as well as warnings to the subject or clinician regarding contraindications or drug interactions. Labels or inserts can include instructions for the clinician or subject for using one or more of the kit components in a method, use, treatment protocol, or therapeutic regimen. Instructions can include dosage amounts, frequency or duration of dose administration, and directions for practicing any of the methods, uses, treatment protocols, or prophylactic or therapeutic regimes described herein. Labels or inserts can include “printed matter,” e.g., paper or cardboard, or separate or affixed to a component, a kit or packing material (e.g., a box), or attached to an ampule, tube, or vial containing a kit component.

The following examples are offered for illustrative purposes only and are not intended to limit the scope of the present invention in any way. Indeed, various modifications of the invention in addition to those shown and described herein will become apparent to those skilled in the art from the foregoing description and fall within the scope of the appended claims.

### EXAMPLES

### Example 1: IdeS Variant Optimization

This example illustrates the creation and testing of IdeS variants with improved properties compared to wildtype IdeS.

Over multiple rounds of optimization, single and multiple substitution variants were designed based on the published crystal structure of wildtype IdeS protein (Wenig et. al. PNAS Dec. 14, 2004 101 (50) 17371-76). After expressing and purifying the variants, the proteins were tested to identify those having improved thermal stability and/or potency compared to wildtype. Results of these screens are shown in Table 3, in which the position numbers of substituted amino acids are in reference to wildtype IdeS precursor protein (SEQ ID NO: 1), including the naturally occurring secretion signal. Corresponding SEQ ID NOs are listed in Table 1. Of the optimized variant proteins, at least those designated GBT-IdeS-0037, GBT-IdeS-0045, and GBT-IdeS-0085, demonstrated both greater thermal stability and potency relative to wildtype IdeS. The variants and control wildtype IdeS (designated GBT-NCC-0005) were expressed with an amino-terminal Met-Gly (MG) rather than the naturally occuring secretion signal, and a carboxy-terminal His tag for ease of purification (SEQ ID NO:85). ND=not determined.

TABLE 3

| Variant | Substitution | | | | IC50 | IC50% |
|---|---|---|---|---|---|---|
| Name | Mutations Relative | | | | fold | improve |
| (GBT-IdeS- | to Wildtype IdeS | Tm1 | Tonset | IC50 | diff to | over |
| nnnn) | Precursor Protein | (° C.) | (° C.) | (nM) | WT | WT |
| 0042 | L220W | 51.18 | 44.7 | 3.83 | 1.45 | 31.0 |
| 0041 | T53V | 52.54 | 45.2 | 3.89 | 1.43 | 30.0 |
| 0083 | L220W, S334L | 51.22 | 42.3 | 3.89 | 1.43 | 29.9 |
| 0045 | S334L | 55.12 | 48.6 | 3.92 | 1.42 | 29.4 |
| 0094 | T53V, S334L | 52.26 | 43.1 | 4.03 | 1.38 | 27.4 |
| 0082 | F172Y, L220W | 51.75 | 42.9 | 4.16 | 1.34 | 25.1 |
| 0043 | S236A | 50.41 | 44.6 | 4.27 | 1.30 | 23.1 |
| 0014 | T53V, S236A, S334L | 50.29 | 42.6 | 4.30 | 1.29 | 22.5 |
| 0089 | F172Y, L220W, | ND | ND | 4.56 | 1.22 | 17.9 |
| | S334L | | | | | |
| 0085 | F172Y, S334L | ND | ND | 4.63 | 1.20 | 16.6 |
| 0093 | T53V, F172Y | ND | ND | 5.19 | 1.07 | 6.6 |
| 0037 | F172Y | 55.47 | 49.3 | 5.37 | 1.03 | 3.3 |
| GBT-NCC- | N/A | 54.29 | 46.8 | 5.55 | 1.00 | 0.0 |
| 0005 | | | | | | |
| 0058 | T53V, F172Y, L220W | 50.07 | 42.1 | 6.04 | 0.92 | −8.8 |
| 0096 | T53V, F172Y, S334L | ND | ND | 6.09 | 0.91 | −9.8 |
| 0013 | T53V, L220W | 46.34 | 38.3 | 6.24 | 0.89 | −12.4 |
| 0063 | T53V, F172Y, S236A, | ND | ND | 6.27 | 0.89 | −13.0 |
| | S334L | | | | | |
| 0016 | L220W, S236A | 46.98 | 40.9 | 6.63 | 0.84 | −19.4 |
| 0068 | Q113F, E132K, | 54.95 | 49.3 | 6.75 | 0.82 | −21.6 |
| | F172Y, Q217K | | | | | |
| 0038 | S252A | 53.98 | 47 | 6.78 | 0.82 | −22.2 |
| 0025 | Q113F, E132K, | 53.71 | 46.9 | 7.59 | 0.73 | −36.7 |
| | Q217K | | | | | |
| 0044 | N265V | 44.54 | 36.7 | 9.44 | 0.59 | −69.9 |
| 0010 | V74R, 1318K | 49.78 | 45.7 | 10.05 | 0.55 | −81.0 |
| 0012 | N265V, S334L | 45.55 | 37.3 | 11.21 | 0.50 | −101.9 |
| 0039 | 1260P | 54.17 | 45.6 | 11.65 | 0.48 | −109.8 |
| 0053 | F172Y, N265V, | 47.29 | 39.2 | 12.72 | 0.44 | −129.1 |
| | S334L | | | | | |
| 0040 | G324M | 55.67 | 49.5 | 12.81 | 0.43 | −130.7 |
| 0066 | T53V, S236A, | 51.62 | 45.7 | 13.71 | 0.40 | −146.9 |
| | G324M, S334L | | | | | |
| 0095 | T53V, G324M | 53.05 | 44.1 | 14.04 | 0.40 | −152.9 |
| 0071 | Q113F, E132K, | 54.92 | 47.6 | 14.83 | 0.37 | −167.1 |
| | F172Y, Q217K | | | | | |
| 0091 | L220W, G324M, | ND | ND | 15.12 | 0.37 | −172.3 |
| | S334L | | | | | |
| 0084 | L220W, G324M | 52.03 | 43.8 | 15.16 | 0.37 | −173.0 |
| 0087 | G324M, S334L | 55.85 | 47.7 | 15.25 | 0.36 | −174.7 |
| 0048 | V74R, F172Y, 1318K | 54.31 | 47.6 | 15.53 | 0.36 | −179.7 |
| 0098 | T53V, G324M, S334L | 54.04 | 45.1 | 17.21 | 0.32 | −210.0 |
| 0061 | T53V, L220W, | 50.59 | 42.9 | 17.70 | 0.31 | −218.8 |
| | G324M | | | | | |
| 0097 | T53V, F172Y, | ND | ND | 18.57 | 0.30 | −234.5 |
| | G324M | | | | | |
| 0090 | F172Y, L220W, | ND | ND | 19.83 | 0.28 | −257.2 |
| | G324M | | | | | |
| 0088 | F172Y, G324M, | ND | ND | 20.18 | 0.28 | −263.5 |
| | S334L | | | | | |
| 0056 | N265V, G324M, | 47.76 | 41.2 | 21.60 | 0.26 | −289.0 |
| | S334L | | | | | |
| 0099 | T53V, F172Y, | ND | ND | 23.55 | 0.24 | −324.2 |
| | G324M, S334L | | | | | |
| 0092 | F172Y, L220W, | 53.72 | 45.7 | 23.71 | 0.23 | −327.0 |
| | G324M, S334L | | | | | |
| 0051 | V74R, 1318K, G324M | 54.67 | 48.1 | 24.96 | 0.22 | −349.6 |
| 0086 | F172Y, G324M | 56.31 | 48.5 | 28.56 | 0.19 | −414.4 |
| 0036 | V1711 | 53.8 | 46.5 | 44.79 | 0.12 | −706.7 |
| 0077 | F172Y, S252A, | 56.52 | 49.3 | ND | ND | ND |
| | 1260P, G324M | | | | | |
| 0022 | V1711, F172Y, | 56.96 | 49.4 | ND | ND | ND |
| | S252A, I260P, | | | | | |
| | G324M | | | | | |

### Example 2: Stability and Potency of Certain IdeS Variants

This example illustrates improved characteristics of certain IdeS variants compared to wildtype IdeS.

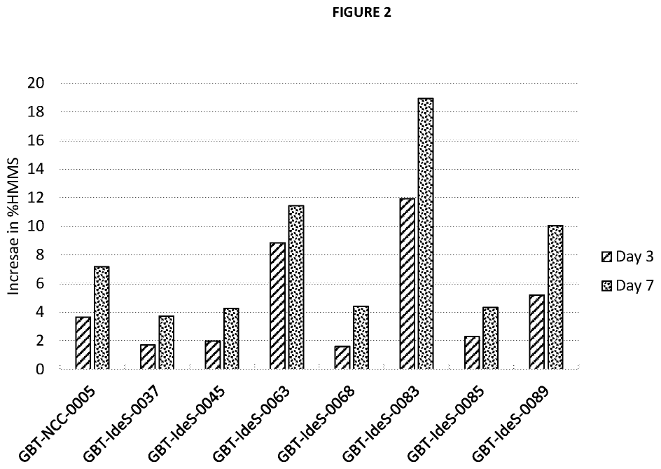

Variant IdeS proteins were generated and stability and potency assessed. Enzyme activity to determine potency was quantified with an activity ELISA assay. Stability was quantified at 37° C. in PBS pH 7.2 (20 mM sodium phosphate, 400 mM NaCl; Neat, 10 microliter injection with samples analyzed in parallel using YMC-Pack Diol-120, 300×8 mm) by differential scanning calorimetry (DSC) after heat-forced degradation. Results are summarized in FIG. 2. The data demonstrate that IdeS variants GBT-IdeS-0037, GBT-IdeS-0045 (same substitution as in PF-07899856), GBT-IdeS-0068, and GBT-IdeS-0085 are more stable than wildtype IdeS (GBT-NCC-0005) at physiological temperature.

Further studies were conducted to compare potency and stability of IdeS variants GBT-IdeS-0045 and GBT-IdeS-0085 compared to GBT-NCC-0005 (similar to wildtype Ides, except that it includes a Met-Gly dipeptide at its N-terminus and a His tag at its C-terminus). Enzyme activity to determine potency was assessed with an activity ELISA assay. Stability was assessed by DSC after heat-forced degradation at 37° C. in PBS pH7.2 formulation for 7 days. Results are summarized in Table 4 and Table 5, below. HMMS=high molecular mass species.

TABLE 4

| | |
|---|---|
| ΔHMMS on | HMMS |
| Day 7 (%) | reduction (%) |

| | |
|---|---|
| IdeS | 37° C. |

| | | |
|---|---|---|
| Wildtype | 7.17 | — |
| GBT-IdeS-0045 | 4.27 | 40.4 |
| GBT-IdeS-0085 | 4.32 | 39.7 |

TABLE 5

| | |
|---|---|
| Potency | Stability |

| IdeS | (nM) | Tₘ (° C.) | Tₒₙₛₑₜ (° C.) |
|---|---|---|---|
| Wildtype | 5.55 | 54.29 | 46.8 |
| GBT-IdeS-0045 | 3.92 | 55.12 | 48.6 |
| GBT-IdeS-0085 | 4.63 | 56.46 | 50 |

Compared to GBT-NCC-0005 (similar to wildtype IdeS), IdeS protein variants GBT-IdeS-0045 and GBT-IdeS-0085 both were more potent towards cleaving IgG, demonstrated greater thermostability, as well as reduced aggregation propensity (or improved colloidal stability) at physiologic temperature in PBS.

### Example 3: Ig Cleavage Specificity of Certain IdeS Variants

This example illustrates that certain IdeS variants specifically cleave IgG, similar to wildtype IdeS.

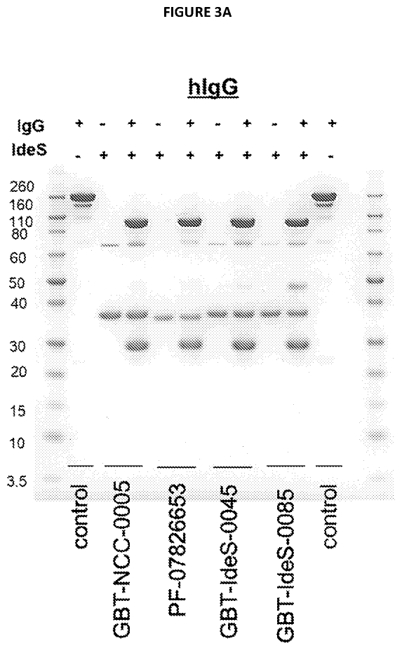

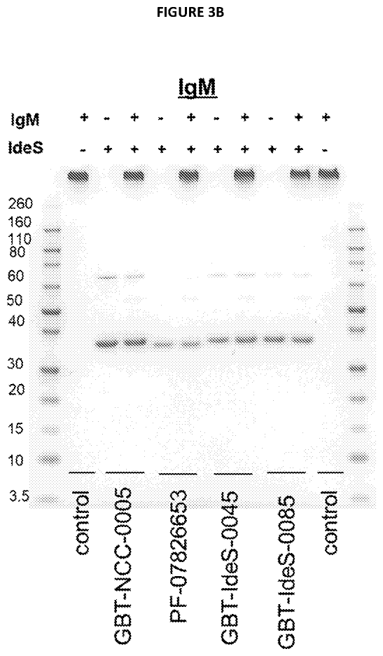

Preparations of purified IgG and IgM proteins were incubated with IdeS variants GBT-IdeS-0045 and GBT-IdeS-0085, as well as IdeS proteins GBT-NCC-0005 and PF-07826653, which are identical to wildtype IdeS in the region corresponding to the mature protein (both were expressed with an N-terminal Met-Gly dipeptide, and GBT-NCC-0005 additionally includes a C-terminal His tag). After incubation, reactions were quenched with SDS buffer and loaded onto denaturing polyacrylamide gels to resolve protein fragments resulting from antibody digestion, followed by Coomassie dye staining using standard methods. Results of digesting IgG proteins are shown in FIG. 3A. Control lanes show protein bands corresponding to intact IgG and IdeS alone. Digestion with wildtype IdeS and each of the IdeS variants converted the single high molecular weight band corresponding to intact IgG into two smaller bands characteristic of F(ab′)2 and Fc fragments. These results demonstrate that, similar to wildtype IdeS, the two IdeS variants tested also efficiently cleaved IgG. Similar experiments were performed testing if wildtype IdeS and IdeS variants had any activity against IgM antibodies, with results shown in FIG. 3B. As expected, wildtype IdeS failed to digest IgM protein and each of the two IdeS variants tested produced similar results. These results demonstrate that IdeS variants GBT-IdeS-0045 and GBT-IdeS-0085 had similar specificity toward binding and cleaving IgG, but not other immunoglobulins (specifically, IgM), as wildtype IdeS.

### Example 4: Wildtype and IdeS Variant Pharmacodynamic Study

This example illustrates the efficacy of an IdeS variant compared to wildtype IdeS in an IgG degradation study in an animal model.

In the study, in vivo potency of an IdeS variant, PF-07899856, was compared to that of PF-07826653, which is identical to wildtype IdeS within the portion corresponding to the mature protein. IdeS variant PF-07899856 has the same substitution relative to wildtype IdeS as the variant GBT-IdeS-0045, but lacks a C-terminal His tag present in the latter. Both PF-07899856 and PF-07826653 were expressed with an amino-terminal Met-Gly dipeptide instead of the native secretion signal peptide, but after expression and purification, the N-terminal Met was missing from the predominant species of both proteins, possibly removed by an endogenous aminopeptidase. The amino acid sequence of PF-07899856 as initally expressed with Met-Gly is provided by SEQ ID NO: 74 and that of PF-07826653 is provided by SEQ ID NO: 72. After removal of the N-terminal Met, the amino acid sequence of PF-07899856 is provided by SEQ ID NO: 75 and that of PF-07826653 is provided by SEQ ID NO:73. Although PF-07826653 differs from mature wildtype IdeS by one or two residues at the amino-terminus, it is referred to in this and Example 5 as “wildtype” for convenience. Male New Zealand white rabbits were dosed with a single dose of either PF-07899856 (1 mg/kg IV, n=3) or wildtype Ides (1 mg/kg IV, n=3), and IgG levels were assessed at various time points subsequent to dosing.

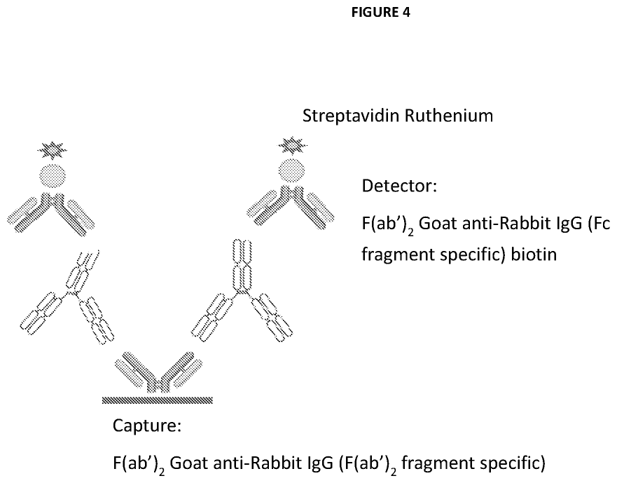

Intact IgG and scIgG were assessed using the Meso Scale Discovery (MSD) assay platform (FIG. 4). In the assay, F(ab′)2 Goat anti-Rabbit IgG specific for F(ab′)2 is used as the capture reagent, and biotinylated F(ab′)2 Goat anti-Rabbit IgG (Fc fragment specific) is used as the detector. Streptavidin ruthenium is used as the detection reagent. This assay detects intact IgG and scIgG and does not detect F(ab′)2 or Fc fragments. For the assay, the standard curve range is 300-0.00508 ng/mL in buffer using purified rabbit IgG control. The upper limit of quantification (ULOQ)=100 ng/ml, and lower limit of quantification (LLOQ)=0.015 ng/ml in buffer. Endogenous QCs (EQCs) were determined in qualification of assay and are summarized in Table 6.

TABLE 6

| Individual Serum | | On Plate Concentration |
|---|---|---|
| Lot # | EQC | (ng/mL) |
| RAB42 | High | 13.83 |
| RAB30 | Mid | 1.2 |
| RAB35 | Low | 0.08 |

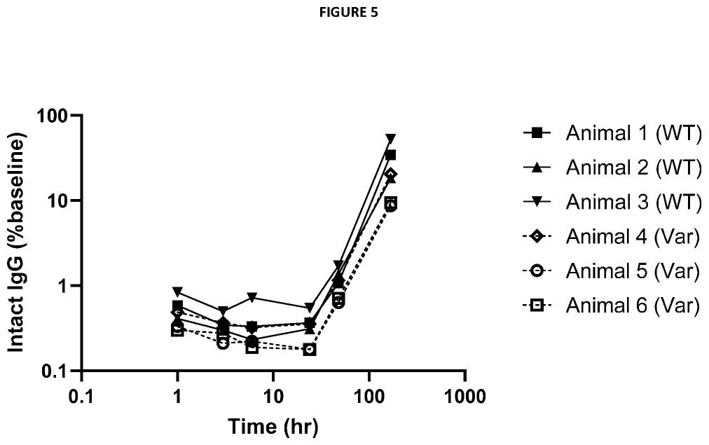

Intact IgG declined with both wildtype IdeS and PF-07899856 treatment, with the mean maximum decrease observed at 6-24 post-dosing (FIG. 5). Animals treated with IdeS variant PF-07899856 had less intact IgG compared to animals treated with wildtype IdeS (FIG. 5). IgG rebound was observed 48 hours and 168 hours post-dosing. Percent efficiency of cleavage by wildtype IdeS (Rabbits 1-3) and IdeS variant PF-07899856 (Rabbits 4-6) is summarized in Table 7.

TABLE 7

| | % Efficiency of IgG cleavage | | | | | |
|---|---|---|---|---|---|---|
| Time | Treatment: Wildtype IdeS (1 mg/kg) | | | Treatment: PF-07899856 (1 mg/kg) | | |
| (hr) | Rabbit 1 | Rabbit 2 | Rabbit 3 | Rabbit 4 | Rabbit 5 | Rabbit 6 |
| 0 | N/A | N/A | N/A | N/A | N/A | N/A |
| 1 | 99.4 | 98.7 | 99.4 | 99.4 | 99.3 | 99.1 |
| 3 | 99.7 | 99.1 | 99.6 | 99.5 | 99.5 | 99.2 |
| 6 | 99.7 | 99.3 | 99.5 | 99.6 | 99.5 | 99.4 |
| 24 | 99.6 | 99.0 | 99.6 | 99.6 | 99.6 | 99.5 |
| 48 | 98.9 | 95.7 | 98.7 | 98.5 | 98.6 | 97.9 |
| 168 | 65.5 | 42.1 | 61.2 | 74.1 | 81.4 | 72.5 |

These results demonstrate that IdeS variant PF-07899856 is more efficacious than wildtype IdeS in decreasing IgG in vivo.

### Example 5: Pharmacokinetics (PK) of IdeS Variants

This example illustrates the PK of IdeS variant on IgG in an animal model study.

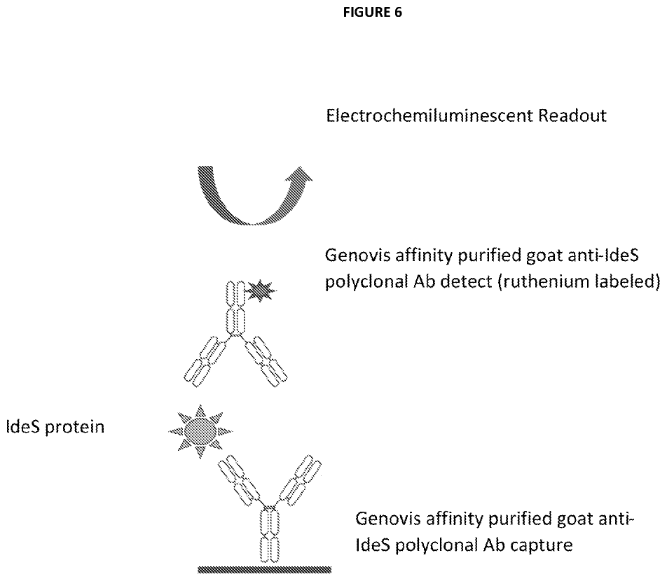

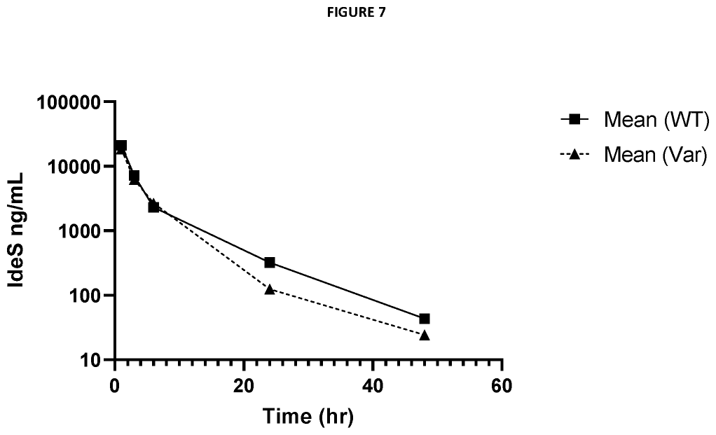

The PK of IdeS variant PF-07899856 and wildtype IdeS were assessed using the Meso Scale Discovery LBA assay (FIG. 6). In this assay, goat anti-IdeS polyclonal antibody is used as the capture reagent, and ruthenium labeled goat anti-IdeS polyclonal antibody is used as the detection reagent. For this assay, the ROQ in 100% was 10.0 ng/ml to 1,280 ng/ml. Tables 8 and 9 provide PK data in male New Zealand white rabbits after a single 1 mg/kg IV dose of wildtype IdeS and IdeS variant PF 07899856, respectively. Time course of IdeS protein plasma concentration after the single dose administration is provided graphically in FIG. 7.

TABLE 8

| Animal | Cₒ | AUC_{0-Tlast} | AUC_{0-inf} | CL | Vₛₛ | Terminal |
|---|---|---|---|---|---|---|
| number | (μg/mL) | (μg*hr/mL) | (μg*hr/mL) | (mL/hr/kg) | (ml/kg) | T_{1/2} (hr) |
| 1 | 29 | 111 | 111 | 9.0 | 44.2 | 7.5 |
| 2 | 38 | 73.8 | 74.0 | 13 | 44.6 | 7.9 |
| 3 | 44 | 115 | 115 | 8.7 | 41.7 | 7.2 |
| Mean | 37 | 99.9 | 100 | 10.4 | 43.5 | 7.5 |

TABLE 9

| Animal | Cₒ | AUC_{0-Tlast} | AUC_{0-inf} | CL | Vₛₛ | Terminal |
|---|---|---|---|---|---|---|
| number | (μg/mL) | (μg*hr/mL) | (μg*hr/mL) | (mL/hr/kg) | (ml/kg) | T_{1/2} (hr) |
| 1 | 31 | 139 | 139 | 7.2 | 30.5 | 6.1 |
| 2 | 33 | 57.0 | 57.2 | 17.5 | 55.9 | 7.3 |
| 3 | 42 | 80.4 | 80.6 | 12.4 | 34.8 | 6.2 |
| Mean | 35 | 92.3 | 92.3 | 12.4 | 40.4 | 6.5 |

These results demonstrate that PK parameters for IdeS variant PF-07899856 and wildtype IdeS were similar.

Although the disclosed teachings have been described with reference to various applications, methods, kits, and compositions, it will be appreciated that various changes and modifications can be made without departing from the teachings herein and the claimed invention below. The foregoing examples are provided to better illustrate the disclosed teachings and are not intended to limit the scope of the teachings presented herein. While the present teachings have been described in terms of these exemplary embodiments, the skilled artisan will readily understand that numerous variations and modifications of these exemplary embodiments are possible without undue experimentation. All such variations and modifications are within the scope of the current teachings.

All references cited herein, including patents, patent applications, papers, text, books, and the like, and the references cited therein, to the extent that they are not already, are hereby incorporated by reference in their entirety. In the event that one or more of the incorporated literature and similar materials differs from or contradicts this application, including but not limited to defined terms, term usage, described techniques, or the like, this application controls.

The foregoing description and Examples detail certain specific embodiments of the invention and describes the best mode contemplated by the inventors. It will be appreciated, however, that no matter how detailed the foregoing may appear in text, the invention may be practiced in many ways and the invention should be construed in accordance with the appended claims and any equivalents thereof.

1. An isolated cysteine protease that specifically cleaves immunoglobulin G (IgG) antibody molecules.

2. The cysteine protease of claim 1, wherein said protease has greater potency or thermal stability relative to wildtype IdeS.

3. The cysteine protease of claim 2, wherein said cysteine protease has a Tₒₙₛₑₜ value,
as determined using differential scanning calorimetry, that is at least or about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4.0° C. greater compared to wildtype IdeS protein, said cysteine protease has a Tₒₙₛₑₜ value, as determined using differential scanning calorimetry, that is at least or about 44.0, 44.1, 44.2, 44.3, 44.4, 44.5, 44.6, 44.7, 44.8, 44.9, 45.0, 45.1, 45.2, 45.3, 45.4, 45.5, 45.6, 45.7, 45.8, 45.9, 46.0, 46.1, 46.2, 46.3, 46.4, 46.5, 46.6, 46.7, 46.8, 46.9, 47.0, 47.1, 47.2, 47.3, 47, 47.5, 47.6, 47.7, 47.8, 47.9, 48.0, 48.1, 48.2, 48.3, 48.4, 48.5, 48.6, 48.7, 48.8, 48.9, 49.0, 49.1, 49.2, 49.3, 49.4, 49.5, 49.6, 49.7, 49.8, 49.9, or 50.0° C.,
said cysteine protease has a T_{M} value, as determined using differential scanning calorimetry, that is at least or about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4.0° C. greater compared to wildtype IdeS protein,
said cysteine protease has a T_{M} value, as determined using differential scanning calorimetry, that is at least or about 51.0, 51.1, 51.2, 51.3, 51.4, 51.5, 51.6, 51.7, 51.8, 51.9, 52.0, 52.1, 52.2, 52.3, 52.4, 52.5, 52.6, 52.7, 52.8, 52.9, 53.0, 53.1, 53.2, 53.3, 53.4, 53.5, 53.6, 53.7, 53.8, 53.9, 54.0, 54.1, 54.2, 54.3, 54.4, 54.5, 54.6, 54.7, 54.8, 54.9, 55.0, 55.1, 55.2, 55.3, 55.4, 55.5, 55.6, 55.7, 55.8, 55.9, 56.0, 56.1, 56.2, 56.3, 56.4, 56.5, 56.6, 56.7, 56.8, 56.9, or 57.0° C. said cysteine protease has IgG cleaving potency, as determined using ELISA and expressed as an IC50 value, that is at least or about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, or 2.5 nM less compared to wildtype IdeS, or
said cysteine protease has IgG cleaving potency, as determined using ELISA and expressed as an IC50 value, that is at most 5.5, 5.4, 5.3, 5.2, 5.1, 5.0, 4.9, 4.8, 4.7, 4.6, 4.5, 4.4, 4.3, 4.2, 4.1, 4.0, 3.9, 3.8, or 3.7 nM.

4-8. (canceled)

9. The cysteine protease of claim 2, wherein the amino acid sequence of said cysteine protease comprises, consists essentially of, or consists of amino acid numbers 1-312 of any one of SEQ ID NOs: 2-71, or amino acid numbers 2-312 of any one of SEQ ID NOs: 2-71, er amino acid numbers 3-312 of any one of SEQ. ID NOs: 3-71.

10. The cysteine protease of claim 9, wherein the amino acid sequence of said cysteine protease comprises, consists essentially of, or consists of the amino acid sequence of any one of SEQ ID NOs: 72-82.

11. A pharmaceutical composition comprising the cysteine protease of claim 1 and a pharmaceutically acceptable carrier.

12. A method of treating a subject in need of treatment or prevention of a disease or disorder characterized by an excess of IgG antibodies, comprising administering to said subject an amount of the cysteine protease of claim 10 that is effective to reduce the concentration of IgG antibodies in a bodily fluid of said subject.

13. The method of claim 12, wherein (i) the bodily fluid is blood, plasma, or serum (ii) said cysteine protease acts by degrading, digesting, or inactivating said IgG antibodies or (iii) the disease or disorder is an autoimmune disease or disorder, and wherein administering said cysteine protease is effective to treat or prevent said autoimmune disease or disorder.

14. (canceled)

15. (canceled)

16. The method of claim 13, wherein an effective amount of said cysteine protease is a dose of at least or about 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.35, 0.40, 0.45, or 0.50 mg/kg subject body weight, wherein said treatment is effective to reduce the concentration of total IgG in the subject's bodily fluid by at least or about 60%, 65%, 70%, 75%, 80%, 85%, 90%, 91%, 92%, 93%, 94%, 95%, 96%, 97%, 98%, 99%, or 100%, or wherein said treatment is effective to reduce the concentration of total IgG in the subject's bodily fluid to not more than about 7, 6.5, 6, 5.5, 5, 4.5, 4, 3.5, 3, 2.5, 2.4, 2.3, 2.2. 2.1, 2.0, 1.9, 1.8, 1.7, 1.6, 1.5, 1.4, 1.3, 1.2, 1.1, 1.0, 0.9, 0.8, 0.7, 0.6, 0.5, 0.4, 0.3, 0.2, 0.1, 0.05 grams per liter.

17. (canceled)

18. (canceled)

19. A method of treating a sensitized subject in need of transplantation of a tissue or organ, comprising administering to said subject an amount of the cysteine protease of claim 10 that is effective to reduce the concentration of anti-HLA antibodies in a bodily fluid of said subject sufficiently to prevent antibody-mediated rejection of said tissue or organ after transplantation.

20. The method of claim 19, wherein (i) the bodily fluid is blood, plasma, or serum (ii) said cysteine protease acts by degrading, digesting, or inactivating said anti-HLA antibodies, (iii) the organ is kidney, liver, heart, pancreas, lung, or intestine, (iv) said subject exhibits a calculated panel reactive antibody assay score of at least or about 10%, 15%, 20%, 25%, 30%, 35%, 40%, 45%, 50%, 55%, or 60%, (v) an effective amount of said cysteine protease is a dose of at least or about 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.35, 0.40, 0.45, or 0.50 mg/kg subject body weight, (vi) said treatment is effective to reduce the concentration of anti-HLA antibodies in the subject's bodily fluid by at least or about 60%, 65%, 70%, 75%, 80%, 85%, 90%, 91%, 92%, 93%, 94%, 95%, 96%, 97%, 98%, 99%, or 100%, (vii) said treatment is effective to reduce the concentration of total IgG in the subject's bodily fluid to not more than about 7, 6.5, 6, 5.5, 5, 4.5, 4, 3.5, 3, 2.5, 2.4, 2.3, 2.2, 2.1, 2.0, 1.9, 1.8, 1.7, 1.6, 1.5, 1.4, 1.3, 1.2, 1.1, 1.0, 0.9, 0.8, 0.7, 0.6, 0.5, 0.4, 0.3, 0.2, 0.1, 0.05 grams per liter, (viii) said treatment is effective to reduce the calculated panel reactive antibody assay score of said subject's serum by at least or about 50%, 55%, 60%, 65%, 70%, 75%, 80%, 85%, 90%, or 95%, or (ix) the subject subsequently undergoes tissue or organ transplantation, and the period between administering the cysteine protease and subsequent transplantation is at least or about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 hours.

21-29. (canceled)

30. A method of treating a subject in need of therapy with a gene therapy vector, comprising administering to said subject an amount of the cysteine protease of claim 10 that is effective to reduce the concentration of IgG antibodies in a bodily fluid of said subject that are specific for a component of said gene therapy vector.

31. The method of claim 30, wherein (i) the bodily fluid is blood, plasma, or serum, (ii) said cysteine protease acts by degrading, digesting, or inactivating said IgG antibodies, (iii) said IgG antibodies are neutralizing antibodies, (iv) said gene therapy vector is a recombinant viral vector.

32-34. (canceled)

35. The method of claim 31, wherein (i) said recombinant viral vector is a recombinant adenoviral vector, recombinant adeno-associated viral vector, or recombinant lentiviral vector, (ii) the titer of neutralizing antibodies is at least or about 1:2, 1:3, 1:4, 1:5, 1:6, 1:7, 1:8, 1:9, 1:10, 1:15, 1:20, 1:25, 1:30, 1:40, 1:50, 1:60, 1:70, 1:80, 1:90, 1:100, 1:120, 1:125, 1:150, 1:175, 1:200, 1:250, 1:300, 1:350, 1:400, 1:450, 1:500, 1:550, 1:600, 1:650, 1:700, 1:750, 1:800, 1:850, 1:900, 1:950, 1:1000, 1:1100, 1:1200, 1:1300, 1:1400, 1:1500, 1:1600, 1:1700, 1:1800, 1:1900, 1:2000, 1:2100, 1:2200, 1:2300, 1:2400, 1:2500, 1:2600, 1:2700, 1:2800, 1:2900, or 1:3000, (iii) an effective amount of said cysteine protease is a dose of at least or about 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.35, 0.40, 0.45, or 0.50 mg/kg subject body weight (iv) said treatment is effective to reduce the titer of neutralizing antibodies in the subject's bodily fluid by at least or about 60%, 65%, 70%, 75%, 80%, 85%, 90%, 91%, 92%, 93%, 94%, 95%, 96%, 97%, 98%, 99%, or 100%, or (v) said treatment is effective to reduce the reduce the titer neutralizing antibodies in the subject's bodily fluid to a value of not more than 1:10, 1:9, 1:8, 1:7, 1:6, 1:5, 1:4, 1:3, 1:2, 1:1, 1:0.9, 1:0.8, 1:0.7, 1:0.6, 1:0.5, 1:0.4, 1:0.3, 1:0.2, or 1:0.1.

36-39. (canceled)

40. The method of claim 31, wherein said subject is gene therapy treatment naïve, and wherein the subject subsequently undergoes treatment with said gene therapy vector.

41. (canceled)

42. (canceled)

43. The method of claim 40, wherein said gene therapy vector is a recombinant adenoviral vector, recombinant adeno-associated viral vector, or recombinant lentiviral vector.

44. The method of claim 40, wherein (i) the period between administering said cysteine protease and subsequent gene therapy is at least or about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 50, 52, 54, 55, 56, 58, 60, 62, 64, 65, 66, 68, 70, 72, 74, 75, 76, 78, 80, 84, 85, 90, 95, 96, 100, 108, 110, 120, 125, 130, 132, 135, 140, 144, 145, 150, 155, 156, 160, 165, or 168 hours, or 8, 9, 10, 11, 12, 13, or 14 days, (ii) the titer of neutralizing antibodies is at least or about 1:2, 1:3, 1:4, 1:5, 1:6, 1:7, 1:8, 1:9, 1:10, 1:15, 1:20, 1:25, 1:30, 1:40, 1:50, 1:60, 1:70, 1:80, 1:90, 1:100, 1:120, 1:125, 1:150, 1:175, 1:200, 1:250, 1:300, 1:350, 1:400, 1:450, 1:500, 1:550, 1:600, 1:650, 1:700, 1:750, 1:800, 1:850, 1:900, 1:950, 1:1000, 1:1100, 1:1200, 1:1300, 1:1400, 1:1500, 1:1600, 1:1700, 1:1800, 1:1900, 1:2000, 1:2100, 1:2200, 1:2300, 1:2400, 1:2500, 1:2600, 1:2700, 1:2800, 1:2900, or 1:3000, (iii) an effective amount of said cysteine protease is a dose of at least or about 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.35, 0.40, 0.45, or 0.50 mg/kg subject body weight, (iv) said treatment is effective to reduce the titer of neutralizing antibodies in the subject's bodily fluid by at least or about 60%, 65%, 70%, 75%, 80%, 85%, 90%, 91%, 92%, 93%, 94%, 95%, 96%, 97%, 98%, 99%, or 100%, or (v) said treatment is effective to reduce the reduce the titer neutralizing antibodies in the subject's bodily fluid to a value of not more than 1:10, 1:9, 1:8, 1:7, 1:6, 1:5, 1:4, 1:3, 1:2, 1:1, 1:0.9, 1:0.8, 1:0.7, 1:0.6, 1:0.5, 1:0.4, 1:0.3, 1:0.2, or 1:0.1.

45-48. (canceled)

49. The method of claim 31, wherein said subject was treated at least once previously with the same type of gene therapy vector with which said subject is in need of therapy, and wherein the subject subsequently undergoes treatment with said gene therapy vector.

50. (canceled)

51. (canceled)

52. The method of claim 49, wherein said gene therapy vector is a recombinant adenoviral vector, recombinant adeno-associated viral vector, or recombinant lentiviral vector.

53. The method of claim 49, wherein (i) the period between administering said cysteine protease and subsequent gene therapy is at least or about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 50, 52, 54, 55, 56, 58, 60, 62, 64, 65, 66, 68, 70, 72, 74, 75, 76, 78, 80, 84, 85, 90, 95, 96, 100, 108, 110, 120, 125, 130, 132, 135, 140, 144, 145, 150, 155, 156, 160, 165, or 168 hours, or 8, 9, 10, 11, 12, 13, or 14 days, (ii) the titer of neutralizing antibodies is at least or about 1:2, 1:3, 1:4, 1:5, 1:6, 1:7, 1:8, 1:9, 1:10, 1:15, 1:20, 1:25, 1:30, 1:40, 1:50, 1:60, 1:70, 1:80, 1:90, 1:100, 1:120, 1:125, 1:150, 1:175, 1:200, 1:250, 1:300, 1:350, 1:400, 1:450, 1:500, 1:550, 1:600, 1:650, 1:700, 1:750, 1:800, 1:850, 1:900, 1:950, 1:1000, 1:1100, 1:1200, 1:1300, 1:1400, 1:1500, 1:1600, 1:1700, 1:1800, 1:1900, 1:2000, 1:2100, 1:2200, 1:2300, 1:2400, 1:2500, 1:2600, 1:2700, 1:2800, 1:2900, or 1:3000, (iii) an effective amount of said cysteine protease is a dose of at least or about 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.35, 0.40, 0.45, or 0.50 mg/kg subject body weight, (iv) said treatment is effective to reduce the titer of neutralizing antibodies in the subject's bodily fluid by at least or about 60%, 65%, 70%, 75%, 80%, 85%, 90%, 91%, 92%, 93%, 94%, 95%, 96%, 97%, 98%, 99%, or 100%, or (v) said treatment is effective to reduce the reduce the titer neutralizing antibodies in the subject's bodily fluid to a value of not more than 1:10, 1:9, 1:8, 1:7, 1:6, 1:5, 1:4, 1:3, 1:2, 1:1, 1:0.9, 1:0.8, 1:0.7, 1:0.6, 1:0.5, 1:0.4, 1:0.3, 1:0.2, or 1:0.1.

54-57. (canceled)

58. The method of claim 12, wherein said subject is a human subject.

59. The method of claim 12, wherein said cysteine protease is administered parenterally, intravenously, or intra-arterially.

60. (canceled)

61. The method of claim 12, wherein said step of administering the cysteine protease to said subject is repeated at least one time.

62. A kit comprising a container having disposed therein a pharmaceutical composition comprising a cysteine protease, and a label with instructions for performing a method according to claim 12.

63. A polynucleotide encoding the cysteine protease of claim 1.

64. An expression vector comprising the polynucleotide of claim 63.

65. A host cell comprising the expression vector of claim 64.

66. (canceled)

67. A method for making a cysteine protease comprising incubating the host cell of claim 65 under conditions sufficient to express said cysteine protease and purifying the cysteine protease produced thereby.


SEQUENCE LISTING (85 sequences)

SEQ ID NO: 1 - protein, 339 aa, Streptococcus pyogenes
MRKRCYSTSAAVLAAVTLFVLSVDRGVIADSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTN

SEQ ID NO: 2 - protein, 321 aa, synthetic construct
MGDSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTNGGGHHHHHH

SEQ ID NO: 3 - protein, 321 aa, synthetic construct
MGDSFSANQEIRYSEVTPYHVTSVWTKGVTPPANFTQGEDVFHAPYQANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNEGAQVLGLFTLSTGQDSWNQTNGGGHHHHHH

SEQ ID NO: 4 - protein, 321 aa, synthetic construct
MGDSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPKLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGKNSAGKVAISAKEIKEDNIGAQVLGLFTLSTGQDSWNQTNGGGHHHHHH

SEQ ID NO: 5 - protein, 321 aa, synthetic construct
MGDSFSANQEIRYSEVTPYHVTSVWTKGVTPPANFTQGEDVFHAPYQANQGWYDITKTFNGKDDLLCGAATAGNMLHWWFDQNKDQIKRYLEEHPEKQKINFNGEQMFDVKEAIDTKNHQLDSKLFEYFKEKAFPKLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGKNSAGKVAISAKEIKEDNIGAQVLGLFTLSTGQDSWNQTNGGGHHHHHH

SEQ ID NO: 6 - protein, 321 aa, synthetic construct
MGDSFSANQEIRYSEVTPYHVTSVWTKGVTPPANFTQGKDVFHAPYQANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNEGAQVLGLFTLSTGQDSWNQTNGGGHHHHHH

SEQ ID NO: 7 - protein, 321 aa, synthetic construct
MGDSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNEGAQVLGLFTLSTGQDSWNQTNGGGHHHHHH

SEQ ID NO: 8 - protein, 321 aa, synthetic construct
MGDSFSANQEIRYSEVTPYHVTSVWTKGVTPPANFTQGEDVFHAPYQANQGWYDITKTRNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNEGAQVLGLFTLSTGQDSWNQTNGGGHHHHHH

SEQ ID NO: 9 - protein, 321 aa, synthetic construct
MGDSFSANQEIRYSEVTPYHVTSVWTKGVTPPANFTQGEDVFHAPYRANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRKNHVINLWGADFDSNGNLKAIYVTDSDSNASIGMKKYFVGVNSAGKVAISAKEIKEDNEGAQVLGLFTLSTGQDSWNQTNGGGHHHHHH

SEQ ID NO: 10 - protein, 321 aa, synthetic construct
MGDSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGDTPVKEGSKDPRGGIFDAVFTRGDQSKLLTSRHDFKEKNLKEISDLIKKELTEGKHLGLSHTYANVRINHVINLWGADFDSNGNLKAIYVTDSDSNASIGMKKYFVGVNSAGKVAISAKEIKEDNIGAQVLGLFTLSTGQDSWNQTNGGGHHHHHH

SEQ ID NO: 11 - protein, 321 aa, synthetic construct
MGDSFSANQEIRYSEVTPYHVTSVWTKGVTPPANFTQGEDVFHAPYRANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNKGAQVLGLFTLSTGQDSWNQTNGGGHHHHHH

SEQ ID NO: 12 - protein, 321 aa, synthetic construct
MGDSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFCDHVIDMFINGYRLSLTNHGPTPVKEGSKDPRGGIFDAVFTRGDQSKLLTSRHDFKEKNLKEISDLIKKELTEGKALCLSHTYANVRINHVINLWGADFDSNGNLKAIYVTDSDSNASIGMKKYFVGVNSAGKVAISAKEIKEDNIGAQVLGLFTLSTGQDSWNQTNGGGHHHHHH

SEQ ID NO: 13 - protein, 321 aa, synthetic construct
MGDSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVIVLWGADFDSNGNLKAIYVTDSDSNASIGMKKYFVGVNSAGKVAISAKEIKEDNIGAQVLGLFTLSTGQDLWNQTNGGGHHHHHH

SEQ ID NO: 14 - protein, 321 aa, synthetic construct
MGDSFSANQEIRYSEVTPYHVTSVWVKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKWLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTNGGGHHHHHH

SEQ ID NO: 15 - protein, 321 aa, synthetic construct
MGDSFSANQEIRYSEVTPYHVTSVWVKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEIADLIKKELTEGKALGLSHTYANVRINHVINLWGADFDSNGNLKAIYVTDSDSNASIGMKKYFVGVNSAGKVAISAKEIKEDNIGAQVLGLFTLSTGQDLWNQTNGGGHHHHHH

SEQ ID NO: 16 - protein, 321 aa, synthetic construct
MGDSFSANQEIRYSEVTPYHVTSVWVKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVIVLWGADFDSNGNLKAIYVTDSDSNASIGMKKYFVGVNSAGKVAISAKEIKEDNIGAQVLGLFTLSTGQDSWNQTNGGGHHHHHH

SEQ ID NO: 17 - protein, 321 aa, synthetic construct
MGDSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKWLTSRHDFKEKNLKEIADLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTNGGGHHHHHH

SEQ ID NO: 18 - protein, 321 aa, synthetic construct
MGDSFSANQEIRYSEVTPYHVTSVWTKGVTPPANFTQGEDVFHAPYVANQGWYDITKTFNGKDDLLCGAATAGMMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVVDSDSNASIGMKKYFVGVNSAGKVAISAKEIKEDNIGAQVLGLFTLSTGQDSWNQTNGGGHHHHHH

SEQ ID NO: 19 - protein, 321 aa, synthetic construct
MGDSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGIYPDHVIDMFINGYRLSLTNHGPTPVKEGSKDPRGGIFDAVFTRGDQSKLLTSRHDFKEKNLKEISDLIKKELTEGKALGLAHTYANVRPNHVINLWGADFDSNGNLKAIYVTDSDSNASIGMKKYFVGVNSAGKVAISAKEIKEDNIGAQVLMLFTLSTGQDSWNQTNGGGHHHHHH

SEQ ID NO: 20 - protein, 321 aa, synthetic construct
MGDSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGYPPDHVIDMFINGYRLSLTNHGPTPVKEGSKDPRGGIFDAVFTRGDQSKLLTSRHDFKEKNLKEISDLIKKELTEGKALGLVHTYANVRVNHVINLWGADFDSNGNLKAIYVTDSDSNASIGMKKYFVGVNSAGKVAISAKEIKEDNIGAQVLSLFTLSTGQDSWNQTNGGGHHHHHH

SEQ ID NO: 21 - protein, 321 aa, synthetic construct
MGDSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINKNGEQMFDVKEAIDTKNHQLDSKLFEYFKEKAFPYYSTKHLGVFPDHVIDMFINGYRLSLTNHGPTPVKEGSKDPRGGIFDAVFTRGDQSKLLTSRHDFKEKNLKEISDLIKKELTEGKALGLSHTEANVRINHVINLWGADFDSNGNLKAIYVTDSDSNASIGMKKYFVGVNSAGKVAISAKEIKEDNIGAQVLGLFTLSTGQDSWNQTNGGGHHHHHH

SEQ ID NO: 22 - protein, 321 aa, synthetic construct
MGDSFSANQEIRYSEVTPYHVTSVWTKGVTPPANFTQGEDVFHAPYVANQGWYDITKTFNGKDDLLCGAATAGNMLHWWFDQNKDFIKRYLEEHPEKQKINFNGKQMFDVKEAIDTKNHQLDSKLFEYFKEKAFPYLSTKHLGVFPDHVIDMFINGYRLSLTNHGPTPVKEGSKDPRGGIFDAVFTRGDKSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTNGGGHHHHHH

SEQ ID NO: 23 - protein, 321 aa, synthetic construct
MGDSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTENHQLDSKLFEYFKEKAFPYLSTKHLGVFPDHVIDMFINGYRLSLTNHGPTPVKEGSKDPRGGIFDAVFTRGDQSKLLTSRHDFKEKNLHEISDLIKKELTEGKALGLSHTYANVRINHVINLWGADFDSNGNLKAIYVTDSDSNASIGMKKYFVGVNSAGKVAISAKEIKKDNIGAQVLGLFTLSTGADSWNQTNGGGHHHHHH

SEQ ID NO: 24 - protein, 321 aa, synthetic construct
MGDSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFGGEQMFDVKNAIDTKNHQLDSKLFEYFKEKAFPYLSTKHLGVFPDHVIDMFINGYRLSLTNHGPTPVKEGSKDPRGGIFDAVFTRGKQSKLLTSRHDFKEKNLKEISDLIKYELLEGKALGLSHTYANVRINHVINLWGADFDSNGNLKAIYVTDSDSNASIGMKKYFVGVNSAGKVAISAKEIKEDNIGAQVLGLFTLSTGQDSWNQTNGGGHHHHHH

SEQ ID NO: 25 - protein, 321 aa, synthetic construct
MGDSFSANQEIRYSEVTPYHVTSVWTKGVTPPANFTQGEDVFHAPYQANQGWYDITKTFNGKDDLLCGAATAGNMLHWWFDQNKDQIKRYLEEHPEKQKINFNGKQMFDVKEAIDTKNHQLDSKLFEYFKEKAFPKLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGKNSAGKVAISAKEIKEDNIGAQVLGLFTLSTGQDSWNQTNGGGHHHHHH

SEQ ID NO: 26 - protein, 321 aa, synthetic construct
MGDSFSANQEIRYSEVTPYHVTSVWTKGVTPPANFTQGEDVFHAPYQANQGWYDITKTFNGKDDLLCGAATAGNMLHWWFDQNKDQIKRYLEEHPEKQKINKNGEQMFDVKEAIDTKNHQLDSKLFEYFKEKAFPYYSTKHLGVFPDHVIDMFINGYRLSLTNHGPTPVKEGSKDPRGGIFDAVFTRGDQSKLLTSRHDFKEKNLKEISDLIKKELTEGKALGLSHTEANVRINHVINLWGADFDSNGNLKAIYVTDSDSNASIGMKKYFVGVNSAGKVAISAKEIKEDNIGAQVLGLFTLSTGQDSWNQTNGGGHHHHHH

SEQ ID NO: 27 - protein, 321 aa, synthetic construct
MGDSFSANQEIRYSEVTPYHVTSVWTKGVTPPANFTQGEDVFHAPYQANQGWYDITKTFNGKDDLLCGAATAGNMLHWWFDQNKDQIKRYLEEHPEKQKINFNGEQMFDVKEAIDTKNHQLDSKLFEYFKEKAFPKLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVNINHVINLWGADFDSNGNLKAIYVTDSDSNASIGMKKYFVGVNSAGKVAISAKEIKEDNIGAQVLGLFTLSTGQDSWNQTNGGGHHHHHH

SEQ ID NO: 28 - protein, 321 aa, synthetic construct
MGDSFSANQEIRYSEVTPYHVTSVWTKGVTPPANFTQGKDVFHAPYQANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVIVLWGADFDSNGNLKAIYVTDSDSNASIGMKKYFVGVNSAGKVAISAKEIKEDNEGAQVLGLFTLSTGQDLWNQTNGGGHHHHHH

SEQ ID NO: 29 - protein, 321 aa, synthetic construct
MGDSFSANQEIRYSEVTPYHVTSVWTKGVTPPANFTQGEDVFHAPYRANQGWYDITKTFNGKDDLLCGAATAGNMLHWWFDQNKDQIKRYLEEHPEKQKINENGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRKNHVIVLWGADFDSNGNLKAIYVTDSDSNASIGMKKYFVGVNSAGKVAISAKEIKEDNIGAQVLGLFTLSTGQDLWNQTNGGGHHHHHH

SEQ ID NO: 30 - protein, 321 aa, synthetic construct
MGDSFSANQEIRYSEVTPYHVTSVWVKGVTPPANFTQGKDVFHAPYQANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEIADLIKKELTEGKALGLSHTYANVRINHVINLWGADFDSNGNLKAIYVTDSDSNASIGMKKYFVGVNSAGKVAISAKEIKEDNEGAQVLGLFTLSTGQDSWNQTNGGGHHHHHH

SEQ ID NO: 31 - protein, 321 aa, synthetic construct
MGDSFSANQEIRYSEVTPYHVTSVWVKGVTPPANFTQGEDVFHAPYQANQGWYDITKTRNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEIADLIKKELTEGKALGLSHTYANVRINHVINLWGADFDSNGNLKAIYVTDSDSNASIGMKKYFVGVNSAGKVAISAKEIKEDNEGAQVLGLFTLSTGQDSWNQTNGGGHHHHHH

SEQ ID NO: 32 - protein, 321 aa, synthetic construct
MGDSFSANQEIRYSEVTPYHVTSVWVKGVTPPANFTQGEDVFHAPYRANQGWYDITKTFNGKDDLLCGAATAGNMLHWWFDQNKDQIKRYLEEHPEKQKINENGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEIADLIKKELTEGKALGLSHTYANVRKNHVINLWGADFDSNGNLKAIYVTDSDSNASIGMKKYFVGVNSAGKVAISAKEIKEDNIGAQVLGLFTLSTGQDSWNQTNGGGHHHHHH

SEQ ID NO: 33 - protein, 321 aa, synthetic construct
MGDSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGIFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTNGGGHHHHHH

SEQ ID NO: 34 - protein, 321 aa, synthetic construct
MGDSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVYPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTNGGGHHHHHH

SEQ ID NO: 35 - protein, 321 aa, synthetic construct
MGDSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLAHTYANVRINHVINLWGADFDSNGNLKAIYVTDSDSNASIGMKKYFVGVNSAGKVAISAKEIKEDNIGAQVLGLFTLSTGQDSWNQTNGGGHHHHHH

SEQ ID NO: 36 - protein, 321 aa, synthetic construct
MGDSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRPNHVINLWGADFDSNGNLKAIYVTDSDSNASIGMKKYFVGVNSAGKVAISAKEIKEDNIGAQVLGLFTLSTGQDSWNQTNGGGHHHHHH

SEQ ID NO: 37 - protein, 321 aa, synthetic construct
MGDSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLMLFTLSTGQDSWNQTNGGGHHHHHH

SEQ ID NO: 38 - protein, 321 aa, synthetic construct
MGDSFSANQEIRYSEVTPYHVTSVWVKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTNGGGHHHHHH

SEQ ID NO: 39 - protein, 321 aa, synthetic construct
MGDSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKWLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTNGGGHHHHHH

SEQ ID NO: 40 - protein, 321 aa, synthetic construct
MGDSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEIADLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTNGGGHHHHHH

SEQ ID NO: 41 - protein, 321 aa, synthetic construct
MGDSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVIVLWGADFDSNGNLKAIYVTDSDSNASIGMKKYFVGVNSAGKVAISAKEIKEDNIGAQVLGLFTLSTGQDSWNQTNGGGHHHHHH

SEQ ID NO: 42 - protein, 321 aa, synthetic construct
MGDSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDLWNQTNGGGHHHHHH

SEQ ID NO: 43 - protein, 321 aa, synthetic construct
MGDSFSANQEIRYSEVTPYHVTSVWTKGVTPPANFTQGEDVFHAPYRANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVYPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNKGAQVLGLFTLSTGQDSWNQTNGGGHHHHHH

SEQ ID NO: 44 - protein, 321 aa, synthetic construct
MGDSFSANQEIRYSEVTPYHVTSVWTKGVTPPANFTQGEDVFHAPYRANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNKGAQVLMLFTLSTGQDSWNQTNGGGHHHHHH

SEQ ID NO: 45 - protein, 321 aa, synthetic construct
MGDSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVYPDHVIDMFINGYRLSLTNHGPTPVKEGSKDPRGGIFDAVFTRGDQSKLLTSRHDFKEKNLKEISDLIKKELTEGKALGLSHTYANVRINHVIVLWGADFDSNGNLKAIYVTDSDSNASIGMKKYFVGVNSAGKVAISAKEIKEDNIGAQVLGLFTLSTGQDLWNQTNGGGHHHHHH

SEQ ID NO: 46 - protein, 321 aa, synthetic construct
MGDSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVIVLWGADFDSNGNLKAIYVTDSDSNASIGMKKYFVGVNSAGKVAISAKEIKEDNIGAQVLMLFTLSTGQDLWNQTNGGGHHHHHH

SEQ ID NO: 47 - protein, 321 aa, synthetic construct
MGDSFSANQEIRYSEVTPYHVTSVWVKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVYPDHVIDMFINGYRLSLTNHGPTPVKEGSKDPRGGIFDAVFTRGDQSKWLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTNGGGHHHHHH

SEQ ID NO: 48 - protein, 321 aa, synthetic construct
MGDSFSANQEIRYSEVTPYHVTSVWVKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKWLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLMLFTLSTGQDSWNQTNGGGHHHHHH

SEQ ID NO: 49 - protein, 321 aa, synthetic construct
MGDSFSANQEIRYSEVTPYHVTSVWVKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVYPDHVIDMFINGYRLSLTNHGPTPVKEGSKDPRGGIFDAVFTRGDQSKLLTSRHDFKEKNLKEIADLIKKELTEGKALGLSHTYANVRINHVINLWGADFDSNGNLKAIYVTDSDSNASIGMKKYFVGVNSAGKVAISAKEIKEDNIGAQVLGLFTLSTGQDLWNQTNGGGHHHHHH

SEQ ID NO: 50 - protein, 321 aa, synthetic construct
MGDSFSANQEIRYSEVTPYHVTSVWVKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEIADLIKKELTEGKALGLSHTYANVRINHVINLWGADFDSNGNLKAIYVTDSDSNASIGMKKYFVGVNSAGKVAISAKEIKEDNIGAQVLMLFTLSTGQDLWNQTNGGGHHHHHH

SEQ ID NO: 51 - protein, 321 aa, synthetic construct
MGDSFSANQEIRYSEVTPYHVTSVWTKGVTPPANFTQGEDVFHAPYVANQGWYDITKTFNGKDDLLCGAATAGNMLHWWFDQNKDFIKRYLEEHPEKQKINFNGKQMFDVKEAIDTKNHQLDSKLFEYFKEKAFPYLSTKHLGVYPDHVIDMFINGYRLSLTNHGPTPVKEGSKDPRGGIFDAVFTRGDKSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTNGGGHHHHHH

SEQ ID NO: 52 - protein, 321 aa, synthetic construct
MGDSFSANQEIRYSEVTPYHVTSVWTKGVTPPANFTQGEDVFHAPYVANQGWYDITKTFNGKDDLLCGAATAGNMLHWWFDQNKDFIKRYLEEHPEKQKINFNGKQMFDVKEAIDTKNHQLDSKLFEYFKEKAFPYLSTKHLGVFPDHVIDMFINGYRLSLTNHGPTPVKEGSKDPRGGIFDAVFTRGDKSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLMLFTLSTGQDSWNQTNGGGHHHHHH

SEQ ID NO: 53 - protein, 321 aa, synthetic construct
MGDSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVYPDHVIDMFINGYRLSLTNHGPTPVKEGSKDPRGGIFDAVFTRGDQSKLLTSRHDFKEKNLKEISDLIKKELTEGKALGLAHTYANVRPNHVINLWGADFDSNGNLKAIYVTDSDSNASIGMKKYFVGVNSAGKVAISAKEIKEDNIGAQVLMLFTLSTGQDSWNQTNGGGHHHHHH

SEQ ID NO: 54 - protein, 321 aa, synthetic construct
MGDSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVYPDHVIDMFINGYRLSLTNHGPTPVKEGSKDPRGGIFDAVFTRGDQSKWLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTNGGGHHHHHH

SEQ ID NO: 55 - protein, 321 aa, synthetic construct
MGDSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKWLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDLWNQTNGGGHHHHHH

SEQ ID NO: 56 - protein, 321 aa, synthetic construct
MGDSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKWLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLMLFTLSTGQDSWNQTNGGGHHHHHH

SEQ ID NO: 57 - protein, 321 aa, synthetic construct
MGDSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVYPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDLWNQTNGGGHHHHHH

SEQ ID NO: 58 - protein, 321 aa, synthetic construct
MGDSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVYPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLMLFTLSTGQDSWNQTNGGGHHHHHH

SEQ ID NO: 59 - protein, 321 aa, synthetic construct
MGDSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLMLFTLSTGQDLWNQTNGGGHHHHHH

SEQ ID NO: 60 - protein, 321 aa, synthetic construct
MGDSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVYPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLMLFTLSTGQDLWNQTNGGGHHHHHH

SEQ ID NO: 61 - protein, 321 aa, synthetic construct
MGDSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVYPDHVIDMFINGYRLSLTNHGPTPVKEGSKDPRGGIFDAVFTRGDQSKWLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDLWNQTNGGGHHHHHH

SEQ ID NO: 62 - protein, 321 aa, synthetic construct
MGDSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVYPDHVIDMFINGYRLSLTNHGPTPVKEGSKDPRGGIFDAVFTRGDQSKWLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLMLFTLSTGQDSWNQTNGGGHHHHHH

SEQ ID NO: 63 - protein, 321 aa, synthetic construct
MGDSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKWLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLMLFTLSTGQDLWNQTNGGGHHHHHH

SEQ ID NO: 64 - protein, 321 aa, synthetic construct
MGDSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVYPDHVIDMFINGYRLSLTNHGPTPVKEGSKDPRGGIFDAVFTRGDQSKWLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLMLFTLSTGQDLWNQTNGGGHHHHHH

SEQ ID NO: 65 - protein, 321 aa, synthetic construct
MGDSFSANQEIRYSEVTPYHVTSVWVKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVYPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTNGGGHHHHHH

SEQ ID NO: 66 - protein, 321 aa, synthetic construct
MGDSFSANQEIRYSEVTPYHVTSVWVKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDLWNQTNGGGHHHHHH

SEQ ID NO: 67 - protein, 321 aa, synthetic construct
MGDSFSANQEIRYSEVTPYHVTSVWVKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLMLFTLSTGQDSWNQTNGGGHHHHHH

SEQ ID NO: 68 - protein, 321 aa, synthetic construct
MGDSFSANQEIRYSEVTPYHVTSVWVKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVYPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDLWNQTNGGGHHHHHH

SEQ ID NO: 69 - protein, 321 aa, synthetic construct
MGDSFSANQEIRYSEVTPYHVTSVWVKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVYPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLMLFTLSTGQDSWNQTNGGGHHHHHH

SEQ ID NO: 70 - protein, 321 aa, synthetic construct
MGDSFSANQEIRYSEVTPYHVTSVWVKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLMLFTLSTGQDLWNQTNGGGHHHHHH

SEQ ID NO: 71 - protein, 321 aa, synthetic construct
MGDSFSANQEIRYSEVTPYHVTSVWVKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVYPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLMLFTLSTGQDLWNQTNGGGHHHHHH

SEQ ID NO: 72 - protein, 312 aa, synthetic construct
MGDSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTN

SEQ ID NO: 73 - protein, 311 aa, synthetic construct
GDSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTN

SEQ ID NO: 74 - protein, 312 aa, synthetic construct
MGDSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDLWNQTN

SEQ ID NO: 75 - protein, 311 aa, synthetic construct
GDSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDLWNQTN

SEQ ID NO: 76 - protein, 310 aa, synthetic construct
DSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDLWNQTN

SEQ ID NO: 77 - protein, 312 aa, synthetic construct
MGDSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVYPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDLWNQTN

SEQ ID NO: 78 - protein, 311 aa, synthetic construct
GDSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVYPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDLWNQTN

SEQ ID NO: 79 - protein, 310 aa, synthetic construct
DSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVYPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDLWNQTN

SEQ ID NO: 80 - protein, 312 aa, synthetic construct
MGDSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVYPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTN

SEQ ID NO: 81 - protein, 311 aa, synthetic construct
GDSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVYPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTN

SEQ ID NO: 82 - protein, 310 aa, synthetic construct
DSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVYPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTN

SEQ ID NO: 83 - protein, 29 aa, Streptococcus pyogenes
MRKRCYSTSAAVLAAVTLFVLSVDRGVIA

SEQ ID NO: 84 - protein, 310 aa, Streptococcus pyogenes
DSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTN

SEQ ID NO: 85 - protein, 9 aa, synthetic construct
GGGHHHHHH